(19) United States
(12) Patent Application Publication
(10) Pub. No.: US 2024/0180484 A1
(43) Pub. Date: Jun. 6, 2024

(54) IMPLANTABLE MEDICAL DEVICES AND METHODS AND SYSTEMS FOR USE THEREWITH FOR MONITORING ARTERIAL BLOOD PRESSURE

(71) Applicant: Pacesetter, Inc., Sylmar, CA (US)

(72) Inventors: Xing Pei, Thousand Oaks, CA (US); Gene A. Bornzin, Santa Monica, CA (US); Alexander R. Bornzin, Ventura, CA (US); Jong Gill, Valencia, CA (US); Wenwen Li, Studio City, CA (US)

(73) Assignee: Pacesetter, Inc., Sylmar, CA (US)

(21) Appl. No.: 18/481,043

(22) Filed: Oct. 4, 2023

Related U.S. Application Data
(60) Provisional application No. 63/386,211, filed on Dec. 6, 2022.

(51) Int. Cl. A61B 5/00; A61B 5/0205; A61B 5/021; G16H 40/67; G16H 50/30

(52) U.S. Cl. CPC A61B 5/4836; A61B 5/0205; A61B 5/02125; A61B 5/686; G16H 40/67; G16H 50/30; A61B 5/0295

(57) ABSTRACT

Implantable systems, and methods for use therewith, monitor a patient's arterial blood pressure without requiring an intravascular pressure transducer. A plurality of calibrations factors are stored, each of which is associated with a respective one of a plurality of different postures, activity levels, or HR ranges, or different combinations thereof. A signal indicative of activity of the patient's heart, and a signal indicative of changes in arterial blood volume of the patient are obtained, and a pulse arrival time (PAT) value is determined. A current posture, activity level, and/or HR of the patient is/are determined and used to identify stored calibration factor(s) that correspond thereto. Values indicative of the patient's arterial blood pressure is/are determined based on the PAT value and the stored calibration factor(s) identified based on the patient's current posture, activity level, and/or HR. Such value(s) and/or changes thereto can be used to trigger and/or adjust therapy.

### PRIORITY CLAIM

This application claims priority to U.S. Provisional Patent Application No. 63/386,211, filed Dec. 6, 2022, which is incorporated herein by reference in its entirety.

### FIELD

Embodiments of the present technology relate to implantable medical devices for monitoring a patient's arterial blood pressure, and methods and systems for use therewith.

### BACKGROUND

A person's circulatory system includes both systemic and pulmonary circulation systems. Pulmonary circulation supplies the lungs with blood flow, while the systemic circulation takes care of all the other parts of the body. The heart serves as a pump that keeps up the circulation of the blood. Both the pulmonary and systemic circulatory systems are made up of arteries, arterioles, capillaries, venules and veins. The arteries take the blood from the heart, while the veins return the blood to the heart

Blood pressure is defined as the force exerted by the blood against any unit area of the vessel wall. The measurement unit of blood pressure is millimeters of mercury (mmHg). Pulmonary and systemic arterial pressures are pulsatile, having systolic and diastolic pressure values. The highest recorded pressure reading is called systolic pressure (SP), which results from the active contraction of the ventricle. Although the arterial pressure and indeed flow in the arteries is pulsatile, the total volume of blood in the circulation remains constant. The lowest pressure reading is called diastolic pressure (DP) which is maintained by the resistance created by the smaller blood vessels still on the arterial side of the circulatory system (arterioles). Stated another way, the systolic pressure is defined as the peak pressure in the arteries, which occurs as a result of the ventricular contraction phase of a cardiac cycle. In contrast, the diastolic pressure is the lowest pressure, which occurs at the resting phase of the cardiac cycle. The pulse pressure (PP) reflects the difference between the maximum and minimum pressures measured (i.e., the difference between the systolic pressure and diastolic pressure). The mean arterial pressure (MAP) is the average pressure throughout the cardiac cycle.

Arterial blood pressure, such as mean arterial pressure (MAP), is a fundamental clinical parameter used in the assessment of hemodynamic status of a patient. Mean arterial pressure can be estimated from real pressure data in a variety of ways. Among the techniques that are used, two are presented below. In these formulas, SP is the systolic blood pressure, and DP is diastolic pressure.

MAP₂=(SP+2DP)/3=⅓(SP)+⅔(DP)3 a.

MAP₁=(SP+DP)/2 b.

Systolic pressure and diastolic pressure can be obtained in a number of ways. A common approach is to use a stethoscope, an occlusive cuff, and a pressure manometer. However, such an approach is slow, requires the intervention of a skilled clinician and does not provide continuous readings as it is a measurement of systolic pressure at a single point in time (or over a relatively short period of time) and a measurement of diastolic pressure at another point in time (or over another relatively short period of time). While systolic pressure and diastolic pressure can also be obtained in more automated fashions, it is not always practical to obtain measures of pressure using a cuff and pressure transducer combination, especially if the intention or desire is to implant a sensor that can monitor arterial pressure on a chronic basis.

Another approach for obtaining measures of arterial blood pressure is to use an intravascular pressure transducer. However, an intravascular device may cause problems such as embolization, nerve damage, infection, bleeding and/or vessel wall damage. Additionally, the implantation of an indwelling intravascular pressure transducer would require a highly skilled clinician such as a surgeon, electrophysiologist, or interventional cardiologist. Further, the cost of an intravascular pressure transducer and its implantation may not be covered by insurance, even though the cost of an ICD and/or pacemaker is covered for a patient.

Plethysmography, the measurement of volume of an organ or body part, has a history that extends over 100 years. Photoplethysmography (PPG) uses optical techniques to perform volume measurements, and was first described in the 1930s. While best known for their role in pulse oximetry, PPG sensors have also been used to indirectly measure blood pressure. For example, non-invasive PPG sensors have been used in combination with an inflatable cuff in a device known as Finapres. U.S. Pat. No. 4,406,289 (Wesseling et al.) and U.S. Pat. No. 4,475,940 (Hyndman) are exemplary patents that relate to the Finapres technique. The cuff is applied to a patient's finger, and the PPG sensor measures the absorption at a wavelength specific for hemoglobin. After the cuff is used to measure the individual's mean arterial pressure, the cuff pressure around the finger is then varied to maintain the transmural pressure at zero as determined by the PPG sensor. The Finapres device tracks the intra-arterial pressure wave by adjusting the cuff pressure to maintain the optical absorption constant at all times.

There are a number of disadvantages to the Finapres technique. For example, when there exists peripheral vasoconstriction, poor vascular circulation, or other factors, the blood pressure measured in a finger is not necessarily representative of central blood pressure. Further, maintaining continuous cuff pressure causes restriction of the circulation in the finger being used, which is uncomfortable when maintained for extended periods of time. Accordingly, the Finapres technique is not practical for chronic use. Additionally, because of the need for a pneumatic cuff, a Finapres device cannot be used as an implanted sensor.

Simple external blood pressure monitors also exist, but they do not offer continuous measurement and data logging capability. These devices can be purchased at a drug store, but patient compliance is required to make regular measurements and accurately record the data. Additionally, portable external miniature monitors that automatically log blood pressure data exist, but these devices can only store a day or so of data and require clinician interaction to download and process the measured data.

As is evident from the above description, there is the need for improved devices, systems and methods for monitoring arterial blood pressure, including systolic pressure, diastolic pressure and/or mean arterial pressure.

### SUMMARY

Embodiments of the present technology are directed to systems and methods that monitor a patient's arterial blood pressure and/or changes thereto without requiring an intravascular pressure transducer. Such embodiments may also involve triggering and/or adjusting therapy based value(s) indicative of the patient's arterial blood pressure and/or changes thereto. As will be appreciated from the below discussion, embodiments of the present technology advantageously recognize and take into account that relationships between pulse arrival time (PAT) values determined by a system for a patient, and the patient's arterial blood pressure, can vary based on the patient's posture, activity level, and heart rate (HR). More specifically, embodiments of the present technology advantageously recognize and take into account that determinations of a patient's arterial blood pressure, based on one or more measured PAT values, can be made more accurate by utilizing one or more calibration factors that depend on at least one of the patient's posture, activity level, or HR, or combinations thereof. In other words, embodiments of the present technology provide improvements over prior methods and devices that monitor a patient's arterial blood pressure based on measured PAT values, where such prior methods and devices either did not use any calibration factors, or if they did, used the same one or more calibration factors regardless of the patient's posture, activity level and HR.

In accordance with certain embodiments, a method includes storing a plurality of calibrations factors, each of which is associated with a respective one of a plurality of different postures, or a respective one of a plurality of different activity levels, or a respective one of a plurality of different HR ranges, or a respective one of a plurality of different combinations of at least two of postures, activity levels and HR ranges. The method also includes obtaining a signal indicative of activity of the patient's heart, obtaining a signal indicative of changes in arterial blood volume of the patient, and determining a pulse arrival time (PAT) value by determining a time from a feature of the signal indicative of activity of the patient's heart to a feature of the signal indicative of changes in arterial blood volume of the patient. The method also includes determining at least one of a current posture, or a current activity level, or a current HR of the patient, and identifying one or more of the stored calibration factors that correspond to at least one of the current posture, or the current activity level, or the current HR of the patient, or a combination of at least two of the current posture, the current activity level and the current HR of the patient. The method additionally includes determining one or more values indicative of the patient's arterial blood pressure based on the PAT value and the one or more of the stored calibration factors identified at the identifying step. The method can also include triggering and/or adjusting therapy based on at least one of the one or more values indicative of the patient's arterial blood pressure.

In accordance with certain embodiments, the obtaining the signal indicative of activity of the patient's heart comprises sensing one of an electrogram (EGM) or an electrocardiogram (ECG), and the obtaining the signal indicative of changes in arterial blood volume of the patient comprises sensing one of a photoplethysmography (PPG) signal or an impedance plethysmography (IPG) signal.

In accordance with certain embodiments, the determining at least one of the current posture, or the current activity level, or the current HR of the patient comprises determining the current posture of the patient; the identifying one or more of the stored calibration factors comprises identifying one or more stored calibration factors that correspond to the current posture of the patient; and the determining the one or more values indicative of the patient's arterial blood pressure comprises determining the one or more values indicative of the patient's arterial blood pressure based on the PAT value and at least one of the one or more stored calibration factors that correspond to the current posture of the patient.

In accordance with certain embodiments, the determining at least one of the current posture, or the current activity level, or the current HR of the patient comprises determining the current activity level of the patient; the identifying one or more of the stored calibration factors comprises identifying one or more stored calibration factors that correspond to the current activity level of the patient; and the determining the one or more values indicative of the patient's arterial blood pressure comprises determining the one or more values indicative of the patient's arterial blood pressure based on the PAT value and at least one of the one or more stored calibration factors that correspond to the current activity level of the patient.

In accordance with certain embodiments, the determining at least one of the current posture, or the current activity level, or the current HR of the patient comprises determining the current HR of the patient; the identifying one or more of the stored calibration factors comprises identifying one or more stored calibration factors that correspond to the current HR of the patient; and the determining the one or more values indicative of the patient's arterial blood pressure comprises determining the one or more values indicative of the patient's arterial blood pressure based on the PAT value and at least one of the one or more stored calibration factors that correspond to the current HR of the patient.

In accordance with certain embodiments, the determining at least one of the current posture, the current activity level or the current HR of the patient comprises determining both the current posture and the current HR of the patient; the identifying one or more of the stored calibration factors comprises identifying one or more stored calibration factors that correspond to a combination of the current posture and the current HR of the patient; and the determining the one or more values indicative of the patient's arterial blood pressure comprises determining the one or more values indicative of the patient's arterial blood pressure based on the PAT value and at least one of the one or more stored calibration factors that correspond to the combination of the current posture and the current HR of the patient.

In accordance with certain embodiments, the determining at least one of the current posture, the current activity level or the current HR of the patient comprises determining both the current activity level and the current HR of the patient; the identifying one or more of the stored calibration factors comprises identifying one or more stored calibration factors that correspond to a combination of the current activity level and the current HR of the patient; and the determining the one or more values indicative of the patient's arterial blood pressure comprises determining the one or more values indicative of the patient's arterial blood pressure based on the PAT value and at least one of the one or more stored calibration factors that correspond to the combination of the current activity level and the current HR of the patient.

In accordance with certain embodiments, the determining at least one of the current posture, the current activity level or the current HR of the patient comprises determining both the current posture and the current activity level of the patient; the identifying one or more of the stored calibration factors comprises identifying one or more stored calibration factors that correspond to a combination of the current posture and the current activity level of the patient; and the determining the one or more values indicative of the patient's arterial blood pressure comprises determining the one or more values indicative of the patient's arterial blood pressure based on the PAT value and at least one of the one or more stored calibration factors that correspond to the combination of the current posture and the current activity level of the patient.

In accordance with certain embodiments, the one or more values indicative of the patient's arterial blood pressure comprises one or more of the following: a value indicative of the patient's systolic pressure (SP); a value indicative of the patient's diastolic pressure (DP); a value indicative of the patient's pulse pressure (PP); or a value indicative of the patient's mean arterial (MAP).

In accordance with certain embodiments, the method further comprises obtaining one or more values indicative of the patient's arterial blood pressure from a non-implanted device, and based thereon, determining or updating one or more of the stored calibrations factors. In certain such embodiments, the one or more of the stored calibrations factors, which is/are updated based on the one or more values indicative of the patient's arterial blood pressure obtained from the non-implanted device, is dependent on at least one of the current posture, or the current activity level, or the current HR of the patient, when the one or more values indicative of the patient's arterial blood pressure was obtained from the non-implanted device.

In accordance with certain embodiments, the sensing the signal indicative of changes in arterial blood volume of the patient comprises sensing both a photoplethysmography (PPG) signal and an impedance plethysmography (IPG) signal. In certain such embodiments, the method can also include selecting, based on at least one of the current posture, or the current activity level, or the current HR of the patient, which one of the PPG and the IPG signals is to be used for the determining the PAT value, which is used for the determining the one or more values indicative of the patient's arterial blood pressure. Alternatively, the determining the PAT value comprises determining a first PAT value using the PPG signal and determining a second PAT value using the IPG signal; and the determining the one or more values indicative of the patient's arterial blood pressure comprises determining a first value indicative of the patient's arterial blood pressure based on the first PAT value, and determining a second value indicative of the patient's arterial blood pressure based on the second PAT value. In certain such embodiments, the method further comprises selecting, based on at least one of the current posture, the current activity level or the current HR of the patient, which one of the first and the second values indicative of the patient's arterial blood pressure is to be at least one of stored or used by the IMD. Alternatively, the method includes selecting, based on at least one of the current posture, the current activity level, or the current HR of the patient, one of a plurality of different ways to determine a weighted average of the first and the second values indicative of the patient's arterial blood pressure; and at least one of storing or using the weighted average of the first and the second values indicative of the patient's arterial blood pressure.

Certain embodiments of the present technology are directed to one or more processor readable storage devices having instructions encoded thereon which when executed cause one or more processors to perform a method for monitoring a patient's arterial blood pressure. Example details of such a method are summarized above, and thus, need not be summarized again.

Certain embodiments of the present technology are directed to a system that comprises memory that stores a plurality of calibrations factors each of which is associated with a respective one of a plurality of different postures, or a respective one of a plurality of different activity levels, or a respective one of a plurality of different heart rate (HR) ranges, or a respective one of a plurality of different combinations of at least two of postures, activity levels and HR ranges. The system also includes electrodes and/or one or more sensors configured to sense a signal indicative of activity of the patient's heart, and configured to sense a signal indicative of changes in arterial blood volume of the patient. Additionally, the system includes one or more further sensors configured to output one or more signals indicative of a current posture or a current activity level of the patient. The system also includes one or more controllers configured to determine a pulse arrival time (PAT) value by determining a time from a feature of the signal indicative of activity of the patient's heart to a feature of the signal indicative of changes in arterial blood volume of the patient. The controller(s) is/are also configured to identify one or more of the stored calibration factors that correspond to at least one of the current posture, or the current activity level, or the current HR of the patient, or a combination of at least two of the current posture, the current activity level and the current HR of the patient. Additionally, the controller(s) is/are configured to determine one or more values indicative of the patient's arterial blood pressure based on the PAT value and at least one of the one or more stored calibration factors that correspond to at least one of the current posture, or the current activity level, or the current HR of the patient, or the combination of at least two of the current posture, the current activity level and the current HR of the patient. The controller(s) can also be configured to trigger and/or adjust therapy based on at least one of the one or more values indicative of the patient's arterial blood pressure. In certain embodiments, such a system includes one or more implantable medical devices (IMDs), and/or one or more external devices, which each of the one or more devices including a respective controller. Where the system includes multiple devices, such devices can communicate with one another so that the system can collectively implement embodiments of the present technology described herein.

In accordance with certain embodiments, the electrodes and/or one or more sensors are used to sense both a photoplethysmography (PPG) signal and an impedance plethysmography (IPG) signal, and the controller(s) is/are configured to select, based on at least one of the current posture, or the current activity level, or the current HR of the patient, which one of the PPG and the IPG signals is to be used to determine the PAT value, which is used to determine the one or more values indicative of the patient's arterial blood pressure.

In accordance with certain embodiments, the electrodes and/or one or more sensors are used to sense both a photoplethysmography (PPG) signal and an impedance plethysmography (IPG) signal, and the controller(s) is/are configured to determine a first PAT value using the PPG signal and determine a second PAT value using the IPG signal, and determine a first value indicative of the patient's arterial blood pressure based on the first PAT value, and determine a second value indicative of the patient's arterial blood pressure based in the second PAT value. In certain such embodiments, the controller(s) is/are configured to select, based on at least one of the current posture, the current activity level, or the current HR of the patient, which one of the first and the second values indicative of the patient's arterial blood pressure is to be at least one of stored or used by the system. Alternatively, the controller(s) is/are configured to select, based on at least one of the current posture, the current activity level or the current HR of the patient, one of a plurality of different ways to determine a weighted average of the first and the second values indicative of the patient's arterial blood pressure. The controller(s) is/are also configured to at least one of store or use the weighted average of the first and the second values indicative of the patient's arterial blood pressure.

Additional and alternative embodiments, features and advantages of the present technology will appear from the following description in which the preferred embodiments have been set forth in detail, in conjunction with the accompanying drawings and claims.

### BRIEF DESCRIPTION OF THE FIGURES

FIG. 1A includes exemplary signal waveforms that are used to show the relative timing of various signals, and how an exemplary metric indicative of pulse arrival time (PAT) can be determined in accordance with an embodiment of the present technology.

FIG. 1B includes the same exemplary signal waveforms shown in FIG. 1A, but shows how another metric indicative of PAT can be determined in accordance with an embodiment of the present technology.

FIG. 1C includes the same exemplary signal waveforms shown in FIG. 1A, but shows how still another exemplary metric indicative of PAT can be determined in accordance with an embodiment of the present technology.

FIG. 2A is a high level flow diagram that is used to explain various embodiments of the present technology that can be used to monitor a patient's arterial blood pressure without requiring an intravascular pressure transducer.

FIGS. 2B-2E illustrate examples of look-up-tables (LUTs) that can be used to store various different calibration factors for various different postures, various different activity levels, various different heart rate (HR) ranges, or combinations thereof.

FIG. 3 illustrates an exemplary implantable cardiac stimulation device that includes a PPG sensor, and which can be used to perform various embodiments of the present technology.

FIG. 4 is a simplified block diagram that illustrates possible components of the implantable medical device shown in FIG. 3.

FIG. 5 is a flow diagram that is used to describe how features of a plethysmography signal can be detected in accordance with specific embodiments of the present technology.

FIG. 6A illustrates an exemplary raw PPG signal over 20 seconds.

FIG. 6B illustrates the PPG signal of FIG. 6A after it has been band-passed filtered, which caused a reduction in noise due to respiration, high frequency noise, and motion artifacts.

FIG. 6C is the same as FIG. 6B, but with R-wave markers added as vertical dashed lines.

FIG. 6D is similar to FIG. 6C, but shows the removal of three outlier beats.

FIG. 6E illustrates an averaged PPG signal resulting from ensemble averaging the remaining cycles of FIG. 6D, and illustrates various feature of the PPG signal that can be determined and used with embodiments of the present technology.

### DETAILED DESCRIPTION OF THE PREFERRED EMBODIMENTS

The following description is of the best modes presently contemplated for practicing various embodiments of the present technology. The description is not to be taken in a limiting sense but is made merely for the purpose of describing the general principles of the present technology. The scope of the present technology should be ascertained with reference to the claims. In the description of the present technology that follows, like numerals or reference designators will be used to refer to like parts or elements throughout. In addition, the first digit of a reference number identifies the drawing in which the reference number first appears.

It would be apparent to one of skill in the art that the present technology, as described below, may be implemented in many different embodiments of hardware, software, firmware, and/or the entities illustrated in the figures. Any actual software, firmware and/or hardware described herein is not limiting of the present technology. Thus, the operation and behavior of the present technology will be described with the understanding that modifications and variations of the embodiments are possible, given the level of detail presented herein.

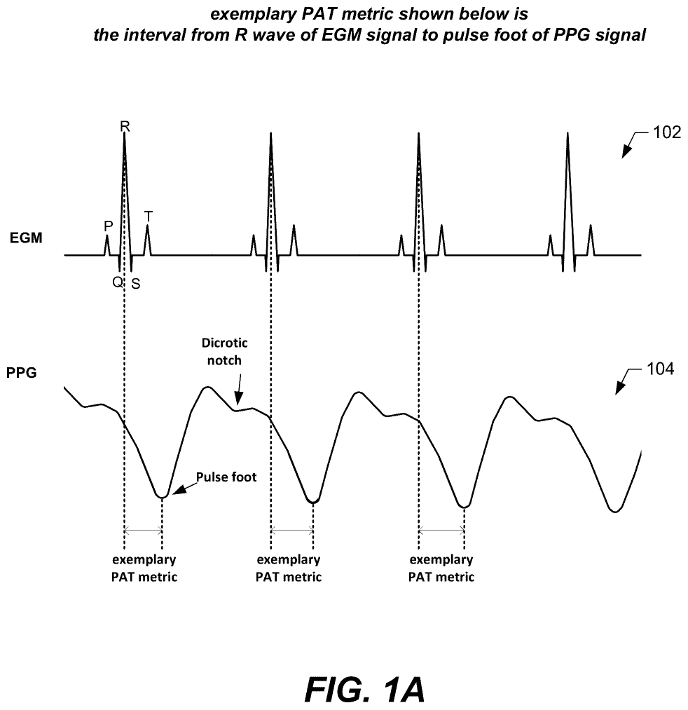

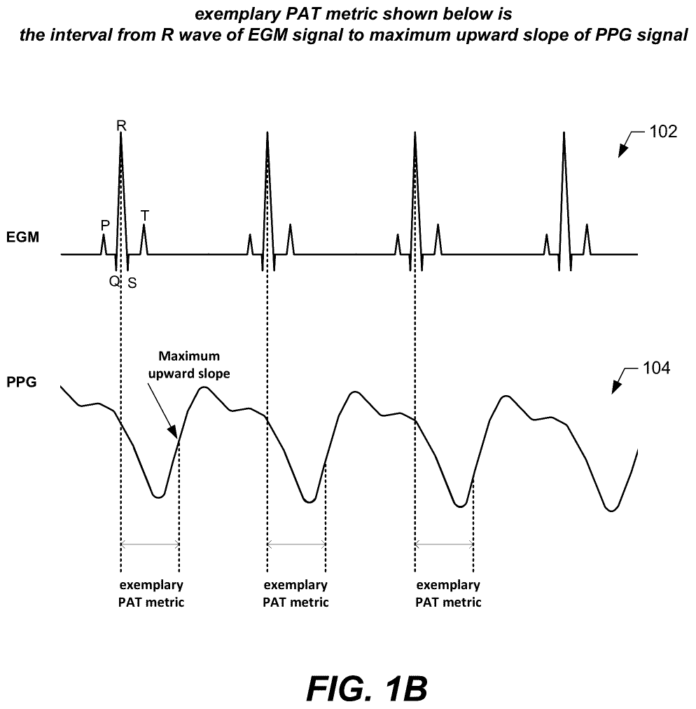

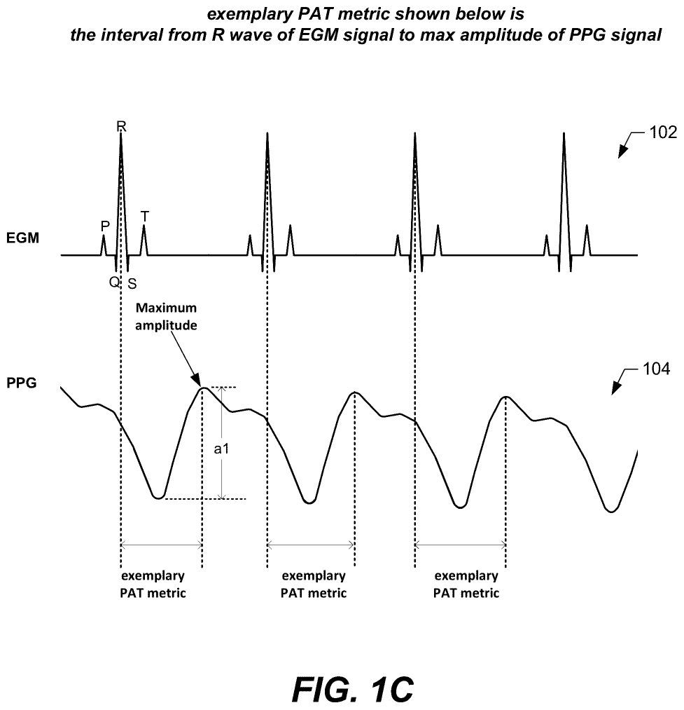

In accordance with specific embodiments of the present technology, an electrogram (EGM), e.g., like 102 in FIGS. 1A-1C, is obtained using implanted electrodes. Additionally, a plethysmography signal, e.g., a photoplethysmography (PPG) signal like 104 in FIGS. 1A-1C, is obtained from an implanted sensor. In accordance with specific embodiments of the present technology, by detecting the relative timing and optionally amplitude of certain features of such signals, various estimates of arterial blood pressure can be obtained, including systolic pressure (SP), diastolic pressure (DP), pulse pressure (PP) and/or mean arterial pressure (MAP).

The SP is the peak pressure in the arteries, which occurs as a result of the ventricular contraction phase of a cardiac cycle. The DP is the lowest pressure in the arteries, which occurs at the end of the resting phase of the arterial circulation. This corresponds to the end of the filling phase of the cardiac cycle with respect to ventricular function. The PP is the difference between the systolic and diastolic pressures. The MAP is a weighted average of pressure throughout the cardiac cycle.

Because implanted electrodes and an implanted sensor are used to estimate arterial blood pressure, a patient's arterial blood pressure can be monitored on a chronic basis. Thus, arterial blood pressure can be estimated multiple times a day to identify blood pressure fluctuations, such as circadian variability. Additionally, arterial blood pressure changes associated with a change in pacing parameters can be monitored.

Additionally, embodiments of the present technology can be used to track a patient's arterial blood pressure to monitor a patient's worsening (or improving) cardiac disease state, and to trigger alerts communicated directly or indirectly to the patient in accordance with clinician established guidelines (e.g., in response to which a patient may be instructed to make a follow-up appointment with the treating clinician or modify the intake of one or more prescription medicines, including one or more blood pressure medications) or transmitted to the treating clinician for review. Additionally, arterial blood pressure estimates can be used as a measure of hemodynamic function.

A plethysmography signal is an example of a signal that is indicative of change in arterial blood volume, and is typically inverted in post-processing. Thus the peak in a plethysmography signal occurs corresponding to the peak in local arterial blood pressure, when blood vessels are distended with larger blood volume. This is because the peak in the plethysmography signal is indicative of the peak wave in arterial blood pressure generated by the patient's heart, as detected by a sensor (e.g., an extravascular PPG sensor) located a distance from the patient's heart. Presuming, e.g., that an extravascular PPG sensor is implanted in the pectoral region of the patient (which is an option, but not necessary), the time it takes from ventricular electrical activation detected by implanted electrodes to develop a pressure wave that travels from the patient's heart to the PPG sensor can be, e.g., on the order of ˜100-300 msec, depending on the location of the electrodes (used to obtain the EGM) and the location of the PPG sensor. The peak pressure wave is initially detectable from an EGM obtained using implanted electrodes. The time at which the peak wave reaches the implanted PPG sensor is detectable from a PPG signal produced by the implanted PPG sensor. Accordingly, the amount of time it takes a peak pulse wave to travel from the patient's heart to the PPG sensor can be determined. Such information has been shown to correlate to arterial blood pressure. It is also possible, and within the scope of the present technology, that the time it takes a peak pulse to travel from the patient's heart to the PPG sensor can be outside the 100-300 msec range mentioned above.

The amount of time it takes for a pulse wave to travel from a patient's heart (and more specifically, their aorta) to a location remote from the patient's heart is often referred to as pulse arrival time (PAT). Embodiments of the present technology use the concept of PAT (also known as pulse transmit time, or pulse wave velocity) to monitor arterial blood pressure.

Referring to FIG. 1A, the representative signal waveforms therein are used to show the relative timing of a signal indicative of activity of the patient's heart and a signal indicative of changes in arterial blood volume remote from the patient's heart. The upper most waveform is an example of an electrogram (EGM) signal 102, which is indicative of activity (and more specifically, electrical activity) of the patient's heart. The lower waveform is an example of a photoplethysmography (PPG) signal 104, which is indicative of changes in arterial blood volume.

Referring to the EGM signal 102, each cycle of the signal 102 is shown as including a P wave, a QRS complex (including Q, R and S waves) and a T wave. The P wave is caused by depolarization of the atria. This is followed by atrial contraction, during which expulsion of blood from the atrium results in further filling of the ventricle. Ventricular depolarization, indicated by the QRS complex, initiates contraction of the ventricles resulting in a rise in ventricular pressure until it exceeds the pulmonary and aortic diastolic blood pressures to result in forward flow as the blood is ejected from the ventricles. Ventricular repolarization occurs thereafter, as indicated by the T wave and this is associated with the onset of ventricular relaxation in which forward flow stops from the ventricles into the aorta and pulmonary arteries. Thereafter, the pressure in the ventricles falls below that in the atria at which time the mitral and tricuspid valves open to begin to passively fill the ventricles during diastole. Depending on the sensing vector(s) used to obtain the EGM signal, some of the above mentioned waves may not be detectable. For example, it may be that only an R-wave is reliably detectable from the EGM signal.

An exemplary metric indicative of pulse arrival time (PAT) is also shown in FIG. 1A. In general, a metric indicative of PAT can be determined, in accordance with embodiments of the present technology, by determining a time from a detected feature of an EGM signal (e.g., 102) to a detected feature of the signal indicative of changes in arterial volume, which can be a PPG signal (e.g., 104), but is not limited thereto. The feature of the signal indicative of cardiac activity is preferably indicative of ventricular depolarization. The feature of the signal indicative of arterial blood volume is preferably indicative of the systolic portion of the signal. In FIG. 1A, the feature of the EGM signal 102 is the R-wave maximum amplitude, and the feature of the PPG signal 104 is the pulse foot that occurs just prior to the start of local blood volume increase. In other words, the metric indicative of PAT can be determined by determining a time from the R-wave peak amplitude of the EGM signal 102 to the pulse foot of the PPG signal 104, as illustrated in FIG. 1A. Alternatively, as illustrated in FIG. 1B, the metric indicative of PAT can be determined by determining a time from the R-wave maximum amplitude of the EGM signal 102 to the maximum upward slope of the PPG signal 104. As illustrated in FIG. 1C the metric indicative of PAT can be determined by determining a time from the R-wave maximum amplitude to the maximum amplitude of the PPG signal 104. A metric indicative of PAT can also be referred to as a PAT metric or a PAT value. Additional or alternative PAT metrics, besides those described with reference to FIGS. 1A-1C, may be determined. For an example, the starting point of the PAT metric can be when the EGM signal 102 equals or exceeds an R-wave threshold, rather than when the R-wave peak occurs.

As described above with reference to FIGS. 1A-1C, a metric indicative of PAT (aka a PAT value) can be determined by determining a time from a detected feature of an EGM signal to a detected feature of the PPG signal. In accordance with an embodiment, a feature of the EGM signal and/or a feature of the PPG signal can be determined using a wavelet transformation, which can provide a consistent and robust technique to detect the feature(s) of the EGM and/or PPG signal used in determining estimates of arterial blood pressure. Wavelet transformation techniques are well known, and thus need not be described herein. As will be described in further detail below, an electrocardiogram (ECG) signal can be used in place of an EGM signal, and/or an impedance plethysmography (IPG) signal can be used in place of a PPG signal.

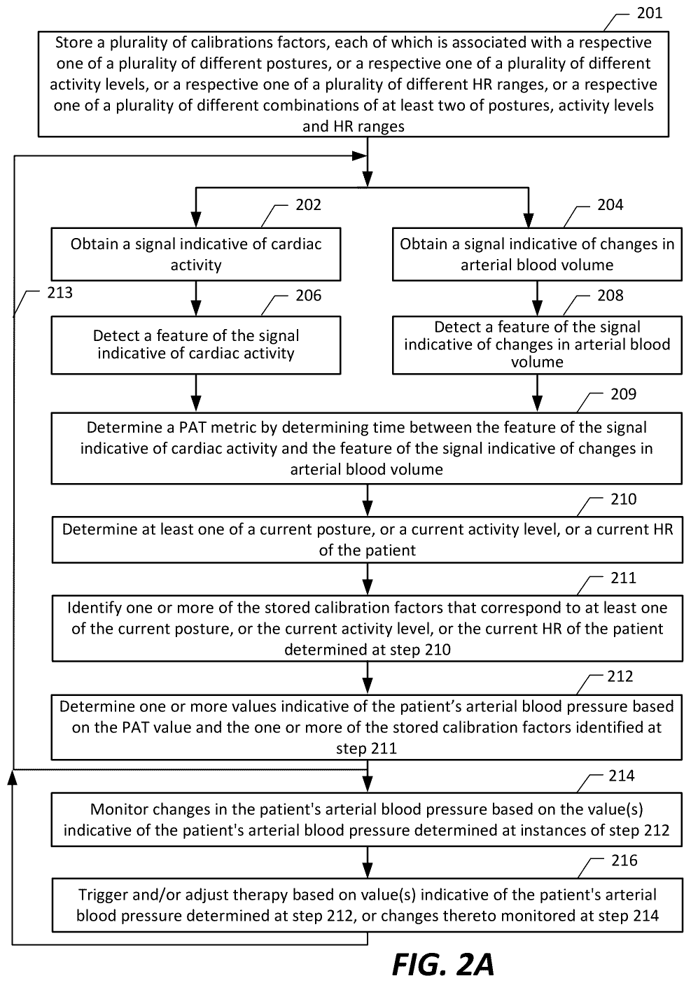

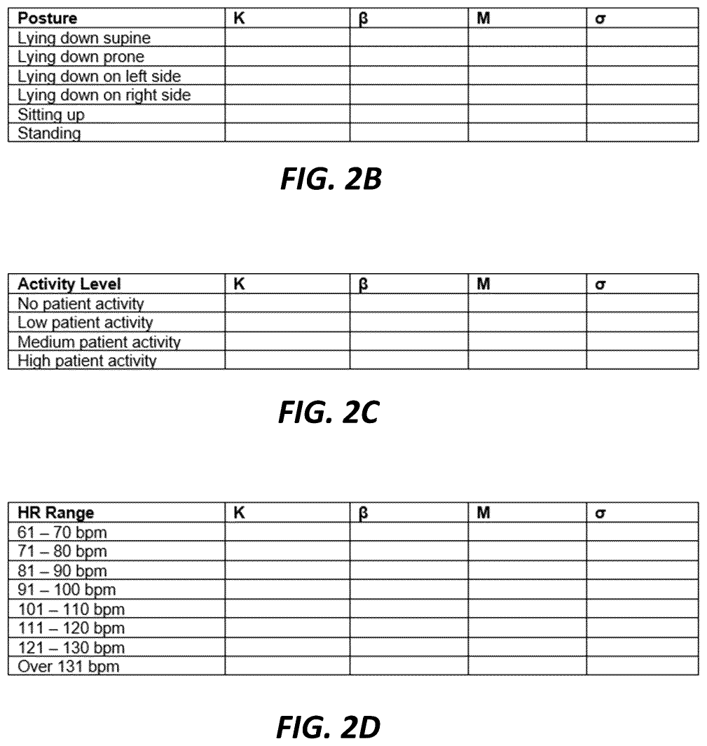

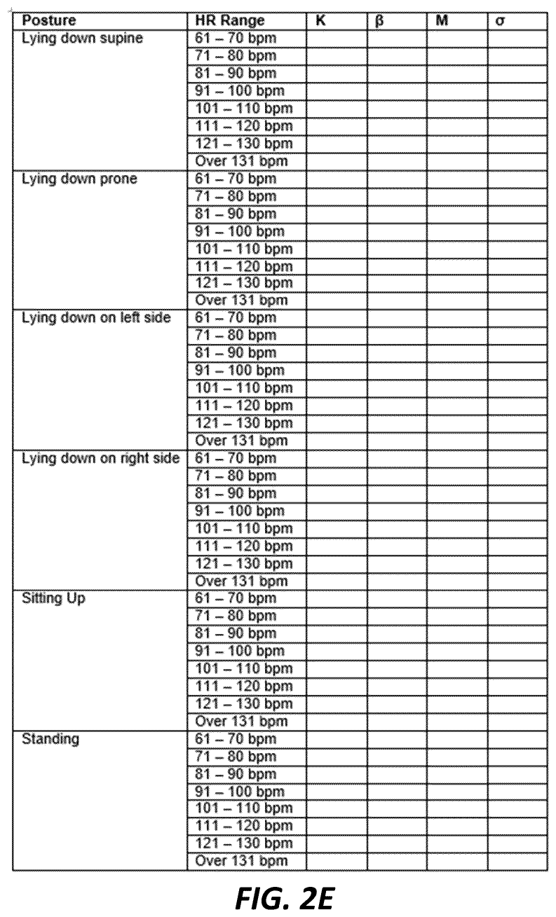

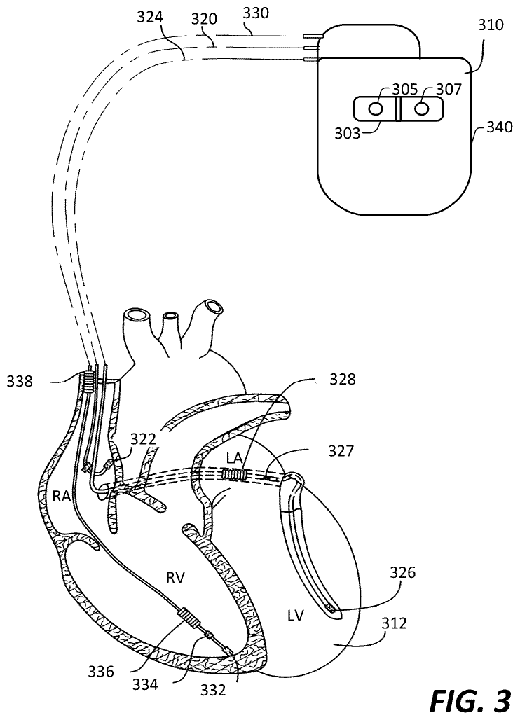

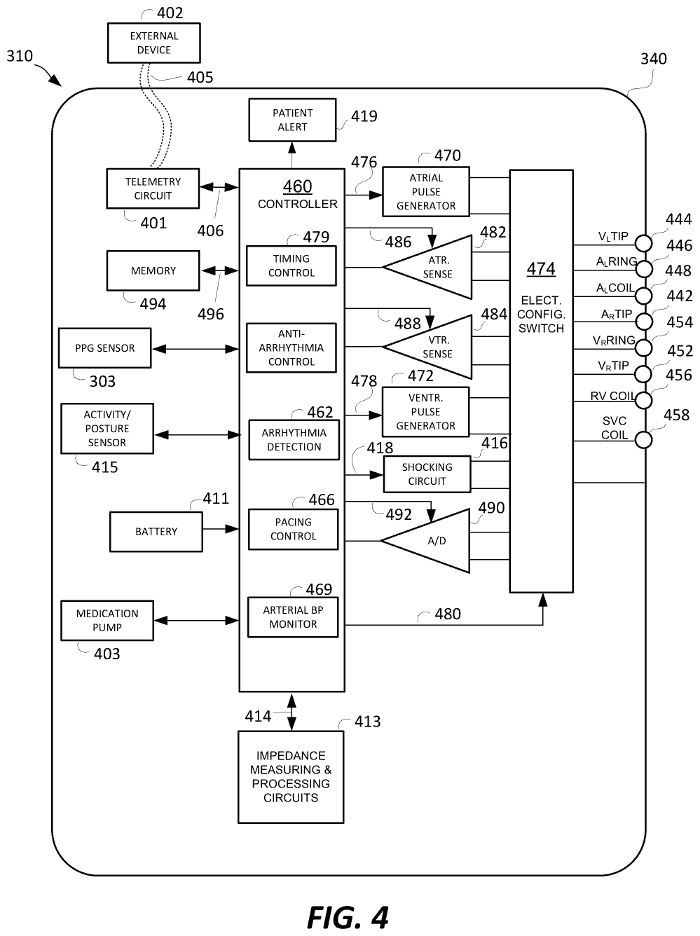

The high level flow diagram of FIG. 2A will now be used to explain various embodiments of the present technology that can be used to monitor a patient's arterial blood pressure without requiring an intravascular pressure transducer. Such embodiments can be implemented by an implantable system, examples of which are discussed below with reference to FIGS. 3 and 4. In FIG. 2A and the other flow diagrams described herein, the various algorithmic steps are summarized in individual ‘blocks’. Such blocks describe specific actions or decisions that are made or carried out as the algorithm proceeds. Where a microcontroller (or equivalent) is employed, the flow diagram presented herein provides the basis for a ‘control program’ that may be used by such a microcontroller (or equivalent) to effectuate the desired control of the implantable system. Those skilled in the art may readily write such a control program based on the flow diagram and other descriptions presented herein.

As will be appreciated from the below discussion, embodiments of the present technology advantageously recognize and take into account that relationships between PAT values determined for a patient, and the patient's arterial blood pressure, can vary based on the patient's posture, activity level, and heart rate (HR). More specifically, embodiments of the present technology advantageously recognize and take into account that determinations of a patient's arterial blood pressure, based on one or more measured PAT values, can be made more accurate by utilizing one or more calibration factors that depend on at least one of the patient's posture, activity level, or HR, or combinations thereof. In other words, embodiments of the present technology described below, provide improvements over prior methods and devices that monitor a patient's arterial blood pressure based on measured PAT values, where such prior methods and devices either do not use any calibration factors, or if they do, use the same one or more calibration factors regardless of the patient's posture, activity level and HR.

Referring to FIG. 2A, step 201 involves storing a plurality of calibrations factors each of which is associated with a respective one of a plurality of different postures, or a respective one of a plurality of different activity levels, or a respective one of a plurality of different HR ranges, or a respective one of a plurality of different combinations of at least two of postures, activity levels and HR ranges. Such calibration factors can be stored in one or more look-up-tables (LUTs), but are not limited thereto. Examples of such LUTs are discussed below with reference to FIGS. 2B-2E.

The different postures can include, for example, lying down supine, lying down prone, lying down on a left side, lying down on a right side, sitting up, and standing, but are not limited thereto. One or more calibration factors can be stored for each such posture. More or less postures than described above can be used. An example LUT that can be used to store various different calibration factors (e.g., K, β, M and σ, discussed in more detail below) for various different posture, is shown in FIG. 2B. More, less and/or different calibrations factors than shown in FIG. 2B can be stored in such a LUT, or in some other manner.

The different activity levels can include, for example, no patient activity, low patient activity, medium patient activity, and high patient activity, but are not limited thereto. One or more calibration factors can be stored for each such activity level. More or less activity levels than described above can be used. An example LUT that can be used to store various different calibration factors (e.g., K, β, M and σ, discussed in more detail below) for various different activity levels, is shown in FIG. 2C. More, less and/or different calibrations factors than shown in FIG. 2C can be stored in such a LUT, or in some other manner.

The different HR ranges can include, for example, a first HR range from 61 to 70 beats per minute (bpm), a second HR range from 71 to 80 bpm, a third HR range from 81 to 90 bpm, a fourth HR range from 91 to 100 bpm, a fifth HR range from 101 to 110 bpm, a sixth HR range from 111 to 120 bpm, a seventh HR range from 121 to 130 bpm, etc., but are not limited thereto. Each HR range can span 10 bpm, as in the above example, or more or less than 10 bpm. It is also possible that some HR ranges have a greater or lesser span than others. One or more calibration factors can be stored for each such HR range. More or less HR ranges than described above can be used. An example LUT that can be used to store various different calibration factors (e.g., K, β, M and σ, discussed in more detail below) for various different HR ranges, is shown in FIG. 2D. More, less and/or different calibrations factors than shown in FIG. 2D can be stored in such a LUT, or in some other manner.

In other words, at step 201, one or more calibration factors can be stored for each of the postures, one or more calibration factors can be stored for each of the activity levels, and/or one or more calibration factors can be stored for each of the HR ranges. Additionally, or alternatively, one or more calibration factors can be stored for each combination of the postures and the HR ranges, for each combination of the activity levels and the HR ranges, for each combination of the activity levels and the HR ranges, and/or for each combination of the postures, the activity levels, and the HR ranges. Shown in FIG. 2E is an example LUT that can be used to store various different calibration factors (e.g., K, β, M and σ, discussed in more detail below) for various different combinations of postures and HR ranges.

It is noted that instead of the HR ranges being defined in terms of beats per minute (bpm), they can equivalently be defined in terms of RR intervals, since there is inverse linear relationship between RR interval and HR, i.e., HR=60/RR interval, and RR interval=60/HR. Accordingly, HR ranges can be quantified in terms beats per minute (bpm), or alternatively, in terms of RR intervals. For example, a first HR range from 61 to 70 bpm can equivalently be defined as corresponding to RR intervals within the range of 0.98 to 0.86 seconds; a second HR range from 71 to 80 bpm can equivalently be defined as corresponding to RR intervals within the range of 0.85 to 0.75 seconds; a third HR range from 81 to 90 bpm can equivalently be defined as corresponding to RR intervals within the range of 0.74 to 0.67; a fourth HR range from 91 to 100 bpm can equivalently be defined as corresponding to RR intervals within the range of 0.66 to 0.60 seconds; a fifth HR range from 101 to 110 bpm can equivalently be defined as corresponding to RR intervals within the range of 0.59 to 0.55 seconds; a sixth HR range from 111 to 120 bpm can equivalently be defined as corresponding to RR intervals within the range of 0.54 to 0.50 seconds; and a seventh HR range from 121 to 130 bpm can equivalently be defined as corresponding to RR intervals within the range of 0.49 to 0.46 seconds, etc., but are not limited thereto.

Examples of the calibration factors stored at step 201 include the calibration factors K, β, M and σ, which are described in more detail below with reference to step 211. One or more of the calibration factors stored at step 201 is/are thereafter identified at step 211 (discussed below), and then used at step 212 (discussed below) to determine one or more values indicative of the patient's arterial blood pressure. The specific types of calibration factors stored at step 201 can depend on the specific equation(s) used at step 212 to determine one or more values indicative of the patient's arterial blood pressure.

An example of an equation used at step 211, to determine a value indicative of the patient's arterial blood pressure based on a PAT value, is: systolic pressure (SP)=K/(PAT value), where K is a calibration factor determined during a calibration procedure. Such a calibration procedure can be performed as part of step 201, or can be performed prior to step 201. For example, during the calibration procedure, while a patient has a specific posture, a specific activity level, or a HR within a specific HR range (or a specific combination of two or more of posture, activity level and HR), an actual value of SP can be determined for the patient using any known accurate acute technique. Additionally, a PAT metric can be measured in one of the manners described herein using an IMD. This will result in K being the only unknown factor in the equation SP=K/(PAT metric), and thus, the calibration factor K can be calculated (e.g., by an external programmer, the IMD, or the like). The value K can then be stored for the specific posture, activity level, or HR (or the specific combination of two or more of posture, activity level and HR). The units of K can be mmHg·msec, so that when K is multiplied by 1/(PAT metric), the resulting SP has units of mmHg.

The patient can then be asked to change at least one of their posture or activity level (which will change their HR), and/or the patient's heart can be paced to change their HR. More generally, while the patient has a new posture, activity level, or HR (or a new combination of two or more of posture, activity level and HR), another actual value of SP can be determined for the patient (using any known accurate acute technique). Additionally, other types of values indicative of arterial blood pressure, such as values of DP, can be determined. More generally, during the calibration procedure, actual measures of arterial blood pressure, such as SP and/or DP, are measured along with PAT values (and optionally peak-to-peak amplitude a1, as will be discussed below), for each of a plurality of different postures, activity levels, or HR ranges, or combinations thereof. The actual measure of the patient's SP and DP can be obtained, e.g., using a non-invasive auscultatory or oscillometric techniques, or an acute invasive intravascular cannula method, or any other acute technique. For a more specific example, actual arterial pressure measurements (SP and DP) can be measured using a high fidelity micronometer-tipped pressure catheter (e.g., model 4F, SPC-120, available from Millar Instruments, Texas), which is temporarily placed in the ascending aorta via a carotid arteriotomy. Other techniques are also possible, and within the scope of the present technology. The above described procedure enables a new value for the calibration factor K (and/or other calibration factor(s)) to be calculated and stored for the new posture, activity level, or HR (or the new combination of two or more of posture, activity level and HR). Multiple iterations of this calibration procedure can be performed, so that a respective value for the calibration factor K (and/or other calibration factor(s)) is stored for each of a plurality of different postures, activity levels, or HR ranges (or for each of a plurality of different combinations of two or more of different postures, activity levels, and HR ranges).

An alternative equation that can be used to determine the patient's systolic pressure is SP=K/(PAT metric)+β. In a similar manner as just described, in addition to determining and storing a respective value for the calibration factor K for each of a plurality of different postures, activity levels, or HR ranges (or for each of a plurality of different combinations of two or more of different postures, activity levels, and HR ranges), a respective value for the calibration factor ß can also be determined and stored for each of a plurality of different postures, activity levels, or HR ranges (or for each of a plurality of different combinations of two or more of different postures, activity levels, and HR ranges).

Alternative and/or additional equations that may be used at step 212 (discussed below) to determine a value indicative of the patient's arterial blood pressure include equations for determining the patient's pulse pressure (PP), such as PP=M·a1, or PP=M·a1+σ. In such equations, a1 is the peak-to-peak amplitude of a signal indicative of changes in arterial blood pressure, which can be obtained at step 204 (discussed below), and M and o are calibration factors that can be determined during a calibration procedure and stored at step 201. More specifically, if the equation PP=M·a1 is to be used at step 212, then a respective value for the calibration factor M can be determined and stored for each of a plurality of different postures, activity levels, or HR ranges (or for each of a plurality of different combinations of two or more of different postures, activity levels, and HR ranges). If the equation PP=M·a1+σ is to be used at step 212, then a respective value for the calibration factor σ can also be determined and stored for each of a plurality of different postures, activity levels, or HR ranges (or for each of a plurality of different combinations of two or more of different postures, activity levels, and HR ranges).

Where calibration factors and/or actual measures of blood pressure are determined by an external device, the calibration factors and/or actual measures of blood pressure can be wirelessly transmitted to the IMD via wireless communication, e.g., such as Bluetooth, or conductive communication, but not limited thereto. The calibration factors and/or actual measures of blood pressure can first be transmitted by the external device to a separate external device configured for secure communication with the IMD and or a remote secure monitoring system. For example the calibration factors and/or actual measures of blood pressure can first be transmitted to an external programmer, a bedside monitor, a smart phone, or a combination thereof, or the like, which can then create secure connection with the IMD to transmit the calibration factors. For a specific example, a bedside monitor may communicate with a smart phone using WiFi, and the smart phone can communicate with the IMD using Bluetooth, such that the smart phone acts as an intermediary for the bedside monitor. Other variations are also possible and within the scope of the embodiments described herein. Calibrations factors can be updated from time to time, e.g., periodically (e.g., once per week, or once per month, but not limited thereto), or in response to one or more triggering events, so as to compensate for maturations of device/tissue interfaces, IMD migration and/or the like.

It is also possible that calibration factors, such as K, β, M and/or σ, but not limited thereto, can be determined for a patient population, or subsets thereof, instead of for individual patients. In other words, the calibrations factors that are stored at step 201 can be patient specific, or can be used for an entire patient population, or for one or more subsets of a patient population.

Still referring to FIG. 2A, step 202 involves obtaining a signal indicative of activity (electrical activity or mechanical activity) of the patient's heart, and step 204 involves obtaining a signal indicative of changes in arterial blood volume of the patient. As can be appreciated from FIG. 2A, steps 202 and step 204 can be performed at the same time. The signal indicative of activity of the patient's heart, obtained at step 202, can be a signal indicative of cardiac electrical activity, such as an electrogram (EGM) or an electrocardiogram (ECG) that is sensed using one or more electrodes implanted within and/or on the patient's heart. It is also possible that an ECG is sensed using subcutaneous electrodes. The signal indicative of activity of the patient's heart, obtained at step 202, can alternative be a signal indicative of cardiac mechanical activity, such as a heart sounds (HS) signal. Such a heart sounds (HS) signal can be sensed using a microphone or an accelerometer (which may or may not be the same accelerometer that is used to detect the patient's posture), but is not limited thereto.

Heart sounds are the noises generated by the beating heart and the resultant flow of blood, and are typically referred to as S1, S2, S3 and S4. The S1 heart sound, which is typically the loudest and most detectable of the heart sounds, is caused by the sudden block of reverse blood flow due to closure of the atrioventricular valves (mitral and tricuspid) at the beginning of ventricular contraction. Isovolumic relaxation (IR) occurs during ventricular diastole and is demarcated approximately by closure of the aortic valve and the second heart sound (S2) and approximately by opening of the mitral valve and the third heart sound (S3), which is more prominent in children and those with abnormal ventricular function when compared to normal adults. The onset of isovolumic relaxation time commences with aortic valve closure, which can be identified by the aortic component (A2) of the second heart sound (S2). The third heart sound (S3) has been linked to flow between the left atrium and the left ventricle, more generally LV filling, and thought to be due to cardiohemic vibrations powered by rapid deceleration of transmitral blood flow. The fourth heart sound (S4) may be present in the late stage of diastole and associated with atrial contraction, or kick, where the final 20% of the atrial output is delivered to the ventricles.

In one embodiment the IMD may determine the electro-mechanical delay between a cardiac event detected in the EGM (e.g., the peak R-wave) and the corresponding detection of the S1 heart sound to determine presence of physiologic factors influencing the choice of signal indicative of cardiac activity to be used in monitoring the arterial blood pressure.

The signal indicative of changes in arterial blood volume of the patient, obtained at step 204, can a photoplethysmography (PPG) signal sensed using an optical sensor of (or coupled to) the IMD, or an impedance plethysmography (IPG) signal sensed using one or more electrodes of (or coupled to) the IMD.

In certain embodiments, an implanted extravascular sensor (e.g., optical sensor, which can also be referred to as a PPG sensor) is used to obtain the PPG signal indicative of changes in arterial blood volume. The signal indicative of changes in arterial blood volume obtained at step 204 can be a PPG signal or some other type of plethysmography signal. An optical sensor can be used to obtain a PPG signal. Examples of electrodes and circuitry that can be used to obtain an EGM signal are discussed below with reference to FIGS. 3 and 4. Exemplary sensors that can be used to obtain a PPG signal are also discussed below with reference to FIGS. 3 and 4.

In still other embodiments, the plethysmography signal indicative of changes in arterial blood volume can be a signal output by a sensor including a piezo-electric diaphragm. Alternative sensors that can be used to produce the plethysmography signal indicative of changes in arterial blood volume, include, but are not limited to, a close range microphone, a sensor including a small mass on the end of a piezo bending beam with the mass located on the surface of a small artery, a transmission mode infrared motion sensor sensing across the surface of a small artery, or a MEMS accelerometer located on the surface of a small artery. Such alternative sensors can be located, e.g., on the tip of a short lead connected to a device that is subcutaneously implanted. The implanted sensor that is used to obtain a plethysmography signal may be implanted close to a patient's aorta. For example, the implanted sensor (used to obtain the signal indicative of changes in arterial blood volume) can be positioned about 10 mm from the patient's aortic root. Such a sensor can alternatively be implanted in the pectoral region of a patient. An alternative location for implantation of the sensor includes, but is not limited to, the patient's abdominal region. It is also possible that the signal indicative of changes in arterial blood volume is an impedance plethysmography signals (IPG) obtained using implanted electrodes (in which case such electrodes can be considered part of a plethysmography sensor). For the remainder of this discussion, it will be assumed that the signal obtained at step 204 is a PPG signal. However, as just explained above, alternative types of plethysmography signals can be used.

Still referring to referring to FIG. 2A, step 206 involves detecting a feature (e.g., a predetermined feature) of the signal indicative of activity of the patient's heart, which signal was obtained at step 202. In an embodiment where the signal obtained at step 202 was an EGM or ECG signal indicative of electrical activity of the patient's heart, the feature of the EGM or ECG signal, detected at step 206, can be a feature indicative of ventricular depolarization, such as a Q-wave, an R-wave and/or an S-wave of the EGM or ECG signal, and/or a QRS complex (e.g., a beginning or a QRS complex, a maximum peak of a QRX complex, a maximum slope of a QRS complect, etc.) of the EGM or ECG signal, but is not limited thereto. However, since the R-wave is the easiest to detect, due to its relatively large magnitude, it is often most practical for ventricular depolarization to be detected by detecting the R-wave. Accordingly, any known or future developed technique for detecting an R-wave (e.g., by peak detection or threshold crossing) can be used to detect ventricular depolarization. Exemplary techniques for detecting R-waves are discussed with reference to FIGS. 4-8 of U.S. Pat. No. 7,403,813, entitled “Systems and Methods for Detection of VT and VF from Remote Sensing Electrodes” (Nabutovsky et al.), which is incorporated herein by reference. Alternatively, other known or future developed techniques for detecting the Q, R and/or S waves can be used to detect ventricular depolarization. More specific examples of the feature that may be detected at step 206 include an R-wave detection (corresponding to an R-wave threshold crossing) or an R-wave peak (corresponding to a maximum amplitude of an R-wave) of an EGM or ECG, but are not limited thereto. In an embodiment where the signal obtained at step 202 was a heart sounds (HS) signal indicative of mechanical activity of the patient's heart, the feature of the HS signal, detected at step 206, can be the beginning of S1, the peak of S1, or the center of mass of S1, but is not limited thereto.

Step 208 involves detecting a feature (e.g., a predetermined feature) of the signal indicative of changes in arterial blood volume of the patient, which signal was obtained at step 204. For the discussion of step 208, it is assumed that the signal obtained at step 204 is a PPG signal. However, as explained above, alternative types of plethysmography signals can be used. In certain embodiments, the feature of the PPG signal detected at step 208 can correspond to a systolic portion of the signal. The morphology of the PPG signal is relatively consistent beat to beat, especially around the systolic portion of the signal. This consistency of the morphology around the systolic portion of the signal can allow for robust and reliable selection of a feature that can serve as a basis for determining a metric indicative of PAT. In accordance with an embodiment, the one or more features of the PPG signal can include the pulse foot of the PPG signal, the pulse peak of the PPG signal, or the maximum positive slope of the PPG signal, but are not limited thereto. In other words, examples of the feature that may be detected at step 208 include the pulse foot of a cycle of a PPG or IPG signal, a maximum upward slope of a cycle of a PPG or IPG signal, or a maximum peak of a cycle of a PPG or IPG signal, but are not limited thereto. As was discussed above, in accordance with certain embodiments, wavelet transformations can be used to detect a feature(s) of the EGM signal and/or PPG signal used in determining the metric indicative of PAT.

Step 209 involves determining a pulse arrival time (PAT) metric by determining a time from the feature of the signal indicative of activity of the patient's heart (detected at step 206) to the feature of the signal indicative of changes in arterial blood volume of the patient (detected at step 208). As noted above, the PAT metric can alternatively be referred to as a metric of PAT, or a PAT value. Further, it is noted that the term pulse arrival time (PAT) is also known as pulse transmit time (PTT) or pulse wave transmit time (PWTT). Referring briefly back to FIGS. 1A, 1B and 1C, the PAT value can be, for example: a time from the R-wave peak amplitude of the EGM signal 102 to the pulse foot of the PPG signal 104, as illustrated in FIG. 1A; a time from the R-wave maximum (aka peak) amplitude of the EGM signal 102 to the maximum upward slope of the PPG signal 104, as illustrated in FIG. 1B; or a time from the R-wave maximum amplitude to the maximum amplitude of the PPG signal 104, as illustrated in FIG. 1C. The type of PAT metric determined at step 209 is preferably the same type determined during the calibration procedure that was used to determine the one or more calibration factors stored at step 201 discussed above.

In certain embodiments it is also useful to determine the peak-to-peak amplitude a1 of the plethysmography signal. One or more peak detection circuit can be used to detect the peak-to-peak amplitude a1. Alternatively, software, hardware and/or firmware can be used to detect the peak-to-peak amplitude a1 based on sample data points of the plethysmography signal, e.g., by determining a difference between maximum and minimum sample values of a plethysmography signal for each cardiac cycle, or a similar algorithm. An exemplary peak-to-peak amplitude a1 is shown in FIG. 1C.

Referring again to FIG. 2A, step 210 involves determining at least one of a current posture, or a current activity level, or a current HR of the patient. Accordingly, at step 210 it is possible that only the current posture, only the current activity level, or only the current HR of the patient is determined. Alternatively, at step 210 it is possible that both the current posture and the current activity level of the patient are determined, or both the current posture and the current HR of the patient are determined, or both the current activity level and the current HR of the patient are determined. Alternatively, at step 210, the current posture, the current activity level, and the current HR of the patient, can all be determined.

One or more accelerometers of, or communicatively coupled to, an IMD, can be used to detect the current posture and/or the current activity level of the patient. More specifically, one or more signals output by one or more accelerometers can be used to determine the current posture of the patient and/or the current activity level of the patient. One or more additional and/or alternative types of sensors, besides accelerometer(s), can be used to determine the current posture and/or the current activity level of the patient, such as a gyroscope, but not limited thereto.

The current HR of the patient can be determined, for example, from the signal obtained at step 202, which as noted above, can be an EGM or ECG signal. Alternatively, the current HR of the patient can be determined from the signal obtained at step 204, which as noted above, can be a PPG or IPG signal, since each cycle of such plethysmography signals generally correspond to an electrical cardiac cycle. In embodiments where the current HR of the patient is determined at step 210 (discussed below), the patient's current HR can be determined in terms of beats per minute (bpm), or equivalently in terms of RR interval, since there is inverse linear relationship between RR interval and HR, as was noted above.

Step 211 involves identifying one or more of the stored calibration factors that correspond to at least one of the current posture, the current activity level, or the current HR of the patient, or a combination of at least two of the current posture, the current activity level and the current HR of the patient. As was mentioned above, in the discussion of step 201, examples of such calibration factors are the calibration factors K, M, β, and σ. In certain embodiment, one or more LUTs, such as those shown in FIGS. 2B-2D, can be used to identify one or more calibration factors at step 211.

Step 212 involves determining one or more values indicative of the patient's arterial blood pressure based on the PAT value(s) and the one or more of the stored calibration factors identified at step 211. In the certain embodiments, where only one PAT value was determined (e.g., the time from an R-wave peak to a peak of a PPG signal) at step 209, that one PAT value is used to estimate arterial blood pressure at step 212. In certain embodiments, the one PAT value can be determined using an EGM (or ECG) signal and a plethysmography signal that are each averaged over multiple cycles, or a respective PAT value can be determined for each of multiple cycles and then averaged to produce the one PAT value.

In other embodiments, more than one type of PAT value may have been determined at step 209, e.g., the time from an R-wave peak to a foot of a PPG signal, and the time from an R-wave peak to a peak of a PPG signal. In such embodiments, at step 212, the multiple types of PAT values can be averaged (equally, or using a weighted average), and an equation and the one or more calibration factors (identified at step 211) can be used to determine the estimate of the patient's arterial blood pressure based on the averaged PAT value. Alternatively, an equation that uses multiple types of PAT values and one or more calibration factors (identified at step 211) can be used to estimate the patient's arterial blood pressure. In such embodiments, the multiple types of PAT values can be determined using an EGM (or ECG) signal and a plethysmography signal that are each averaged over multiple cycles, or the PAT values are determined for each of multiple cycles and then averaged.

At step 212 general estimates of arterial blood volume can be determined, so that changes in such general estimates can be detected. It is also within the scope of the present technology that estimates of SP, DP, PP and/or MAP be determined, as will now be explained. A PAT value is inversely related to SP, in that the greater the PAT value the lower the SP, and the lower the PAT value the greater the SP. As noted above, examples of equations that can be used at step 212 are SP=K/(PAT metric), or SP=K/(PAT metric)+β. In such equations, K and B are examples of calibrations factors stored at step 201 (for various different postures, activity levels, or HR ranges, or combinations thereof), identified at step 211, and used at step 212. In summary, at step 212, SP can be determined based on a PAT metric (determined at step 209), and one or more calibration factors (identified at step 211), and using an equation (e.g., SP=K/PAT metric, or SP=K/(PAT metric)+β), or using a look-up table. Such a look-up table can essentially include pre-calculated estimates of arterial blood pressure for various different combinations of PAT values and calibration factor values, but are not limited thereto.

A value indicative of pulse pressure (PP) can be determined based on the amplitude a1. A value indicative of diastolic pressure (DP) can be determined by subtracting the value indicative of PP from the value indicative of SP (i.e., DP=SP−PP). The value indicative of PP is mainly determined so that the value of DP can be determined. Accordingly, a value of DP can be determined based on the value of SP and the value of a1.

Peak-to-peak amplitude a1 is directly related to the PP, in that the greater a1 the greater the PP, and the lower the a1 the lower the PP. In a simplest embodiment, PP=a1. However, it would be beneficial to use a patient specific calibration factor (e.g., a constant M) when determining PP. In other words, in specific embodiments, PP=M·a1, or possibly PP=M·a1+σ, where M (and possibly also σ) can be determined during a calibration procedure, as will be described below. During calibration, while actual values of SP are being determined for various PAT metrics, and for specific postures, activity levels or HR ranges, or combinations thereof, actual values of DP can also be determined for various values of a1. This will enable the patient specific calibration factor M (and possibly also σ) to be determined during the calibration procedure, for each of a plurality of different postures, activity levels, and/or HR ranges, or combinations thereof. By combining PP=M·a1 with DP=SP−PP, a resulting equation is DP=SP−(M·a1). Since actual values of DP and SP can be obtained during calibration (at implant and/or thereafter), for each of a plurality of postures, activity levels and/or HR ranges, or combinations thereof, and values of a1 can be measured during calibration, a patient specific calibration factor M (and possibly also σ) can be determined and stored for each of the plurality of postures, activity levels and/or HR ranges, or combinations thereof. It is also possible that the calibration factor(s) M and/or σ can be determined, for each of a plurality of postures, activity levels and/or HR ranges, or combinations thereof, for a patient population, or one or more subsets thereof, instead of for individual patients. Other formulas are also possible, and could be derived by determining actual values of the DP for various different values of a1. After implant, in similar manners as were discussed above, an algorithm or look-up table can be used to calculate PP based on a1.

If both SP and DP are estimated, mean arterial pressure (MAP) can also be estimated. For example, the equation MAP=⅓ SP+⅔ DP can be used. Alternatively, the equation MAP=(SP+DP)/2 can be used. Use of other equations are also possible, and within the scope of the present technology.

As indicated by line 213, steps 202, 204, 206, 208, 209, 210, 211 and 212 can be repeated from time to time (e.g., periodically, aperiodically, in response to a triggering event, etc.), with one or more estimates of the patient's arterial blood pressure being determined each time. In specific embodiments these steps are performed multiple times a day, e.g., once per hour or other interval of time.

At step 214, changes in the patient's arterial blood pressure are monitored based on the value(s) indicative of the patient's arterial blood pressure determined at instances of step 212. This can include, e.g., detecting increases or decreases in the patient's arterial blood pressure, or detecting that the patient's arterial blood pressure has not changed. As will be described below, step 214 can also include detecting circadian variability in arterial blood pressure and/or fluctuations over shorter or longer periods.

In accordance with certain embodiments, one or more values indicative of the patient's arterial blood pressure is determined from a non-implanted device, such as a programmer, and based thereon, one or more of the stored calibrations factors are updated. In certain such embodiments, the one or more of the stored calibrations factors, which is/are updated based on the one or more values indicative of the patient's arterial blood pressure obtained from the non-implanted device, is dependent on at least one of the posture, or the activity level, or the HR of the patient, when the one or more values indicative of the patient's arterial blood pressure was obtained from the non-implanted device.

In accordance with certain embodiments, at step 204, both a PPG signal and an IPG signal are obtained. In certain such embodiments, the method can also include selecting, based on at least one of the posture, or the activity level, or the HR of the patient, which one of the PPG and the IPG signals is to be used for the determining the PAT value, which is used for the determining the one or more values indicative of the patient's arterial blood pressure. This could be because it was determined that for certain postures, activity levels, and/or HR ranges, determinations of blood pressure based on PAT values are more accurate using one of the PPG and IPG signals, and for other postures, activity levels and/or HR ranges, determinations of blood pressure based on PAT values are more accurate using the other one of the PPG and IPG signals.

In other embodiments, where both a PPG signal and an IPG signal are obtained at step 204, a first PAT value is determined at step 209 using the PPG signal, and second PAT value is also determined at step 209 using the IPG signal. In certain such embodiments, at step 212 a first value indicative of the patient's arterial blood pressure is determined based on the first PAT value, and a second value indicative of the patient's arterial blood pressure is also determined based on the second PAT value. In certain such embodiments, one of the first and the second values indicative of the patient's arterial blood pressure, which is to be stored and/or used by the IMD, is selected based on at least one of the posture, the activity level, or the HR of the patient. Alternatively, based on at least one of the posture, the activity level, or the HR of the patient, one of a plurality of different ways to determine a weighted average of the first and the second values indicative of the patient's arterial blood pressure is selected and used. The weighted average of the first and the second values indicative of the patient's arterial blood pressure can thereafter be used and/or stored by the IMD. Additionally details of how values indicative of the patient's arterial blood pressure can be stored and/or used by the IMD are discussed below.

Values indicative of the patient's arterial blood pressure, determined instances of step 212, can be stored in the memory of an IMD, so that such values are available for upload to a non-implantable device that can be used by a physician to observe trends over time in the patient's arterial blood pressure. Additionally, or alternatively, values indicative of the patient's arterial blood pressure can be used to trigger notifications. Additionally, or alternatively, if the IMD that performs the method described with reference to FIG. 2A is capable of delivering therapy, then therapy can be triggered and/or adjusted (at step 216) based on values indicative of the patient's arterial blood pressure, determined at instances of step 212. For example, the patient's arterial blood pressure may be purposely modified by adjusting at least one pacing parameter and/or pacing configuration based on the monitored changes in the patient's arterial blood pressure. Various factors that may trigger the adjusting of pacing parameter(s) and/or pacing configuration at step 216 are described below. For example, estimates of arterial blood pressure and/or changes therein can be compared to one or more thresholds to identify when it would be useful to modify the patient's arterial blood pressure to keep it more in line with a desired value or range.

In accordance with certain embodiments, the IMD that determines one or more values indicative of a patient's arterial blood pressure based on a PAT value and one or more of the stored calibration factors is the same as the IMD that triggers and/or adjusts therapy based on at least one of the determined value(s) indicative of the patient's arterial blood pressure. In accordance with other embodiments, where a patient has multiple (i.e., at least two) IMDs, a first IMD (aka IMD1) determines one or more values indicative of the patient's arterial blood pressure based on a PAT value and one or more of the stored calibration factors, and provides (and more specifically, transmits) such information to a second IMD (aka IMD2), and the second IMD triggers and/or adjusts therapy based on at least one of the value(s) indicative of the patient's arterial blood pressure. Such information can be transmitted from the first IMD to the second IMD using radio frequency (RF) communication, conductive communication, or inductive communication, but not limited thereto. More specifically, referring back to FIG. 2A, it is possible that all of the steps shown therein are performed by the same IMD. Alternatively, steps 201 through 214 may be performed by a first IMD (under control of its controller), and step 216 can be performed by a second IMD (under control of its controller). It is also possible that steps 201 through 212 are performed by a first IMD (under control of its controller), and steps 214 and 216 can be performed by a second IMD (under control of its controller). It would also be possible for an external device (e.g., a wearable, or a bedside monitor) to perform one or more of the steps in the flow diagram of FIG. 2A, and for an IMD (or multiple IMDs) to perform one or more other steps in the flow diagram of FIG. 2A. More generally, the methods summarized with reference to the flow diagram of FIG. 2A can be achieved using a distributed processing system. Other variations are also possible and within the scope of the embodiments described herein.

There are numerous reasons why it could be valuable to monitor changes in a patient's arterial blood pressure and to modify a patient's arterial blood pressure. For example, patients with heart failure (HF) and compromised systolic function may supply an inadequate amount of blood to organs of the body. For such patients, it may be desirable to maintain blood pressure. Other patients may have hypertension or compromised diastolic function, and thus have elevated blood pressure. For such patients, it may be desirable to reduce blood pressure. As discussed in further detail below, there are some patients that may have orthostatic hypotension, which is a reduction in blood pressure upon changing from supine to standing position. For such patients, it may be desirable to selectively increase blood pressure in response to a change in body position. Many patients are taking medications to control blood pressure and cardiovascular properties, but may not be in compliance at all times. For such patients, it may be desirable to provide a backup way of controlling blood pressure for those times that the patient is not in compliance. Embodiments of the present technology described herein can be used to achieve these and other goals.

In accordance with certain embodiments, one or more values indicative of a patient's arterial blood pressure can be used in real time in combination with one or more other physiologic parameters to track the patient's heart failure (HF) status, and more generally heart condition. The one or more other physiological parameters can include, but are not limited to, heart rate variability (HRV), heart sounds, cardiac impedance, and/or the like. Example techniques for tracking a patient's HF status, and more generally heart condition, with which values of a patient's arterial blood pressure (or changes therein) can be utilized, are described in U.S. patent application Ser. No. 17/194,354, filed Mar. 8, 2021, and titled “Method and System for Heart Condition Detection Using an Accelerometer,” which is incorporated herein by reference.

In accordance with an embodiment, to increase arterial blood pressure, a pacing rate can be increased and/or an atrio-ventricular (AV) interval can be reduced. Where the implantable system is capable of performing multi-site left ventricular (MSLV) pacing, it may also be possible to increase the patient's arterial blood pressure by pacing at an additional pacing site within the left ventricular chamber. To reduce arterial blood pressure, a pacing rate can be reduced and/or the AV interval (aka AV delay) can be increased. Where the implantable system is capable of performing MSLV pacing, it may also be possible to reduce the patient's arterial blood pressure by stopping pacing at one of multiple pacing sites within the left ventricular chamber. Other pacing intervals can be adjusted to modify the patient's arterial blood pressure, including, but not limited to, interventricular (VV) delay, and intraventricular delay (e.g. LV1-LV2 delay where MSLV pacing is performed). In general, adjustments that increase pumping efficiency should increase arterial blood pressure, and adjustments that decrease pumping efficiency should decrease arterial blood pressure. Other techniques for adjusting pumping efficiency include, but are not limited to, adjusting pacing site locations within the left ventricular chamber. Other types of pacing parameters that may be adjusted based on value(s) of the patient's arterial blood pressure, or changes thereto, include PV delay and a base pacing rate. Low arterial blood pressure may be indicative of non-capture of a patient's left bundle branch. In accordance with certain embodiments, which are for use with patient's having an IMD that delivers septal pacing, an amplitude of septal pacing pulses can be increased in response to a value of the patient's arterial blood pressure falling below a corresponding threshold, in order to improve a probability of achieving left bundle branch capture, which in-turn should result in an increase in the patient's arterial blood pressure.

Use of alternative therapy for increasing or decreasing (and more generally, modifying) the patient's arterial blood pressure are also within the scope of the present technology. For example, additionally, or alternatively, to reduce arterial blood pressure, neurostimulation configured to cause vasodilation can be delivered, and to increase arterial blood pressure neurostimulation configured to restrict blood vessels can be delivered. For another example, if an implantable system is equipped with a medication pump (also known as a drug pump), such a pump can be used to deliver medication to modify arterial blood pressure. Other types of therapy can also be delivered to modify (increase or decrease) arterial blood pressure as needed while still being within the scope of the present technology. Such alternative types of therapy can supplement or supplant the pacing therapy adjustments discussed above.

The circadian variability associated with healthy individuals involves arterial blood pressure rising in the morning and falling in the evening. Abnormalities in this circadian variability have been shown to be an independent risk factor for heart disease and stroke, even in apparently healthy individuals without chronic disease. For example, excessive swings in blood pressure (e.g., beyond an acceptable range) can be indicative of a patient having a high risk for cardiovascular disease, especially where the patient is pre-diabetic. Additionally, extraordinary short term oscillations in blood pressure can be detrimental to patients.

Embodiments of the present technology can be used to monitor a circadian variability of a patient's arterial blood pressure, and to modify the circadian variability if appropriate. More specifically, in accordance with certain embodiments, step 214 includes monitoring a circadian variability of the patient's arterial blood pressure, and another step (step 216) can include adjusting pacing parameter(s) and/or pacing configuration to increase the circadian variability if the circadian variability is below a first threshold, and/or adjusting pacing parameter(s) and/or pacing configuration to decrease the circadian variability if the circadian variability is above a second threshold. Additionally, or alternatively, pacing parameter(s) and/or pacing configuration can be adjusted to cause the circadian variability pattern of the patient's arterial blood pressure to track a predetermined circadian variability pattern. Exemplary ways to increase or reduce arterial blood pressure by adjusting pacing parameters and/or pacing configuration were discussed above, and thus need not be repeated. Alternative types of therapy (some examples of which were discussed above) can also be delivered to modify the circadian variability of a patient's arterial blood pressure, which can supplement or supplant the pacing therapy adjustments.

If the circadian variability of the patient's arterial blood pressure is too large, this can be indicative of disease and/or disease progression. In accordance with an embodiment, metric(s) indicative of how often and/or to what extent the circadian variability of the patient's arterial blood pressure exceeds a threshold can be monitored and tracked, which can be indicative of cardiovascular risk and disease progression. Where such metric(s) exceed thresholds indicative of excessive risk or disease progression, an alarm can be triggered. In accordance with an embodiment, an alert triggering mechanism can be part of an implanted system (e.g., patient alert 419 in FIG. 4). Alternatively or additionally, an implanted system can trigger a non-implanted alarm of a non-implanted system. In still other embodiments, where arterial blood pressure information is transmitted, e.g., via telemetry to an external device, a non-implanted alert can be triggered to thereby notify the patient and/or the patient's physician of cardiovascular risk and/or disease progression. In one embodiment the alert be communicated to an external devices that can then prompt the patient to enter responses to a series of health related questions, e.g., do you have a headache, are you fatigued . . . which can then be wirelessly transmitted to the treating clinician along with the measured BP that instigated the alert and at least in some instances the output of a histogram tracking blood pressure as a function of time. In other embodiments the alert may include other measures indicative of physiologic patient well-being including for example, AF burden, activity, sleep quality, cardiac impedance or the like.

When a patient is inactive a patient's arterial blood pressure should be relatively low, and when the patient is active their arterial blood pressure should be correspondingly higher. This is in part because a person's muscles need more blood to operate efficiently when the patient is active (e.g., exercising). The arterial blood pressure in a patient having chronotropic incompetence may not appropriately adjust to the patient's activity level, which can result in the patient easily becoming fatigued.

Where at least some of the steps of FIG. 2A are triggered in response to detection of various different activity, posture states, or HR ranges, information about the patient's activity, posture and/or HR can also be stored along with estimates of the patient's arterial blood pressure, so that such information can be correlated. In other words, there could be a cross-correlation of estimates of arterial blood pressure with levels of activity, posture and/or HR. This is also possible where the steps of FIG. 2A are not triggered in response to detection of various different activity and/or posture states, so long as activity and/or posture is also monitored.

Embodiments of the present technology are not limited to the exact order and/or boundaries of the steps shown in FIG. 2A. In fact, many of the steps can be performed in a different order than shown, and many steps can be combined, or separated into multiple steps. For another example, certain steps shown in the FIG. 2A can be separated into two or more steps. The only time order is important is where a step acts on the results of a previous step.

In accordance with specific embodiments of the present technology, estimates of arterial blood pressure, including estimates of SP, DP, PP and/or MAP (and/or changes therein) can be stored so that a physician or clinician can upload such measurements when visiting the physician or clinician. More generally, estimates of arterial blood pressure, obtained in accordance with embodiments of the present technology can be used to assess the hemodynamic status of a patient. This can include tracking a patient's cardiac disease state, including but not limited to, heart failure. For example, increases in measures of arterial blood pressure and/or circadian variability over a length of time (e.g., a month) can be interpreted as a worsening of a heart failure condition.

In accordance with various embodiments, because implanted electrodes and an implanted sensor are used to estimate a patient's arterial blood pressure, the patient's arterial blood pressure can be monitored on a chronic basis. Thus, arterial blood pressure and variations (e.g., circadian variations) can be tracked to monitor a patient's evolving cardiac health, and to trigger an alert, therapy and/or adjust therapy based on the estimated arterial blood pressure. FIGS. 3 and 4 will now be used to describe an exemplary implantable system that can be used to implement embodiments of the present technology including but not limited to embodiments that monitor and modify a patient's arterial blood pressure without requiring an intravascular pressure transducer.

### Example Implantable System

Referring to FIG. 3, the implantable system is shown as including an implantable stimulation device 310, which can be a pacing device and/or an implantable cardioverter defibrillator (ICD). One of skill in the art will appreciate that the many of the embodiments of the present technology described herein are not limited to use with implanted devices, active implanted devices or implanted systems. Indeed, many of the embodiments of the present technology described herein may be implemented in a wearable device or a subcutaneous implantable cardiac monitor (ICM), or the like. Accordingly, it should be understood that embodiments of the present technology described herein are not limited to being used with the device 310, or more generally, to be used with or by an implanted device or system. The device 310 is shown as being in electrical communication with a patient's heart 312 by way of three leads, 320, 324 and 330, which can be suitable for delivering multi-chamber stimulation and shock therapy. The leads can also be used to obtain EGM signals, for use in embodiments of the present technology. As described below, it is also possible that one of these leads (or another lead) can include an optical sensor (also referred to as a PPG sensor) that is useful for obtaining a PPG signal, similar to signal 104 shown in FIG. 1.

In FIG. 3, the implantable device 310 is shown as having a PPG sensor 303 (also referred to as an optical sensor) attached to its housing 340. The PPG sensor 303, which can be used to obtain a PPG signal similar to signal 104 shown in FIG. 1, includes a light source 305 and a light detector 307. The light source 305 can include, e.g., at least one light-emitting diode (LED), incandescent lamp or laser diode, but is not limited thereto. The light detector 307 can include, e.g., at least one photoresistor, photodiode, phototransistor, photodarlington or avalanche photodiode, but is not limited thereto. Light detectors are often also referred to as photodetectors or photocells.

The light source 305 outputs light that is reflected or backscattered by surrounding patient tissue, and reflected/backscattered light is received by the light detector 307. In this manner, changes in reflected light intensity are detected by the light detector 307, which outputs a signal indicative of the changes in detected light. The output of the light detector 307 can be filtered and amplified. The signal can also be converted to a digital signal using an analog to digital converter, if the PPG signal is to be analyzed in the digital domain. A PPG sensor can use a single wavelength of light (e.g., infrared (IR) light within the range of 800-960 nm, but not limited thereto), or a broad spectrum of many wavelengths. Additional details of exemplary implantable PPG sensors are disclosed in U.S. Pat. Nos. 6,409,675 and 6,491,639, both entitled “Extravascular Hemodynamic Sensor” (both Turcott), which are incorporated herein by reference.

It is generally the output of the photodetector that is used to produce a PPG signal. However, there exist techniques where the output of the photodetector is maintained relatively constant by modulating the drive signal used to drive the light source, in which case the PPG signal is produced using the drive signal, as explained in U.S. Pat. No. 6,731,967, entitled” Methods and Devices for Vascular Plethysmography via Modulation of Source Intensity,” (Turcott), which is incorporated herein by reference.

The extravascular PPG sensor 302 can be attached to a housing 340 of an implantable device, which as mentioned above can be, e.g., a pacemaker and/or an ICD. Exemplary details of how to attach a sensor module to an implantable cardiac stimulation device are described in U.S. Pat. No. 7,653,434, entitled “Autonomous Sensor Modules for Patient Monitoring” (Turcott et al.), which is incorporated herein by reference. It is also possible that the PPG sensor 302 be integrally part of the implantable cardiac stimulation device 310. For example, the PPG sensor 302 can be located within the housing 340 of an ICD (and/or pacemaker) that has a window through which light can be transmitted and detected. In a specific embodiment, the PPG sensor 302 has a titanium frame with a light transparent quartz or sapphire window that can be welded into a corresponding slot cut in the housing of the ICD. This will insure that the ICD enclosure with the welded PPG sensor will maintain a hermetic condition.

Where the PPG sensor is incorporated into or attached to a chronically implantable device 310, the light source 305 and the light detector 307 can be mounted adjacent to one another on the housing or header of the implantable device, or on the bottom of the device, or at any other location. The light source 305 and the light detector 307 can be placed on the side of the implantable device 310 that, following implantation, faces the chest wall, and are configured such that light cannot pass directly from the source to the detector. The placement on the side of the device 310 that faces the chest wall maximizes the signal to noise ratio by directing the signal toward the highly vascularized musculature, and shielding the source and detector from ambient light that enters the body through the skin. Alternatively, at the risk of increasing susceptibility to ambient light, the light source 305 and the light detector 307 can be placed on the face of the device 310 that faces the skin of the patient. Other variations are also possible.

In an alternative embodiment, the PPG sensor 303 (or other plethysmography sensor) is remote from the housing 340 of the device 310, but communicates with the electronics in the device housing 340 via one or more wires, optical fibers, or wirelessly (e.g., using telemetry, RF signals and/or using body fluid as a communication bus medium). This embodiment enables an obtained PPG signal to be indicative of changes in arterial blood volume at a location remote from the patient's heart, where such location is also remote from the device housing 340. If desired, multiple PPG signals can be obtained, e.g., using multiple PPG sensors at different locations.

In another embodiment, optical fibers can be used to transmit light into and detect light from tissue that is remote from the device housing, even though the light source and light detector are located within or adjacent the device housing 340. This embodiment enables an obtained PPG signal to be indicative of changes in arterial blood volume at a location remote from the patient's heart, where such location is remote from the device housing 340, even though the light source 305 and light detector 307 are not remote from the housing. The distal end of the optical fiber(s) associated with the light source can be generally parallel to the distal end of the optical fiber(s) associated with the light detector, so that the light detector detects the portion of light reflected from tissue. Alternatively, the distal end of the optical fiber(s) associated with the light source can generally face the distal end of the optical fiber(s) associated with the light detector, with tissue therebetween, so that the light detector detects the portion of light transmitted through (as opposed to reflected from) the tissue therebetween.

In an embodiment, a PPG sensor can be within or attached to a lead that may extend from a main device housing 340. Accordingly, in this embodiment, a housing of the sensor module is sized to fit within the implantable lead. For example, the PPG can be located proximal from the distal tip of the lead so that the PPG sensor is sufficiently remote from the heart that variations in pulse transmission time are detectable and meaningful. The portion of the lead that is adjacent to a window of the PPG sensor module, where light is to exit and enter, should allow the light to pass in and out of the sensor. Thus, the lead may be transparent, or include its own window, opening, or the like. The lead can include tines for attaching the lead in its desired position, but may include any other type of fixation means (e.g., a pigtail shaped fixation means), or none at all. The lead can also have a suture sleeve, that enables the lead to be sutured to patient tissue. Additional details of a lead that includes an optical sensor that can be used to produce a PPG signal are provided in U.S. Pat. No. 7,660,616, entitled “Improved Multi-Wavelength Implantable Oximeter Sensor” (Poore), and U.S. Pat. No. 7,840,246, entitled “Implantable Device with a Calibration Photodetector” (Poore), which are incorporated herein by reference.

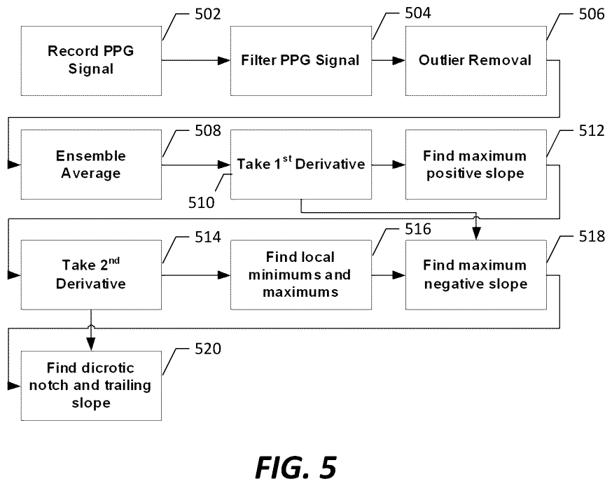

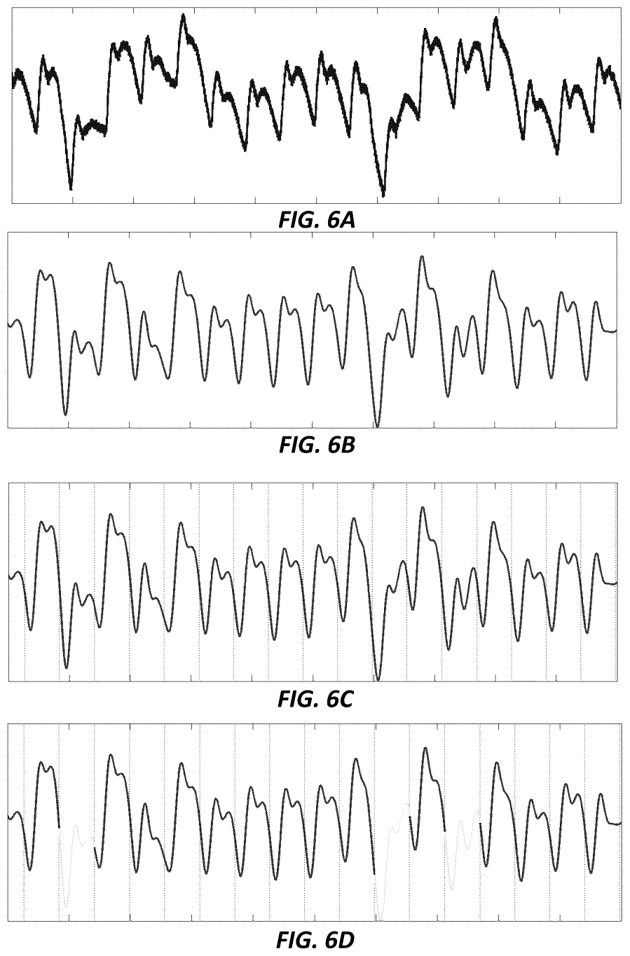

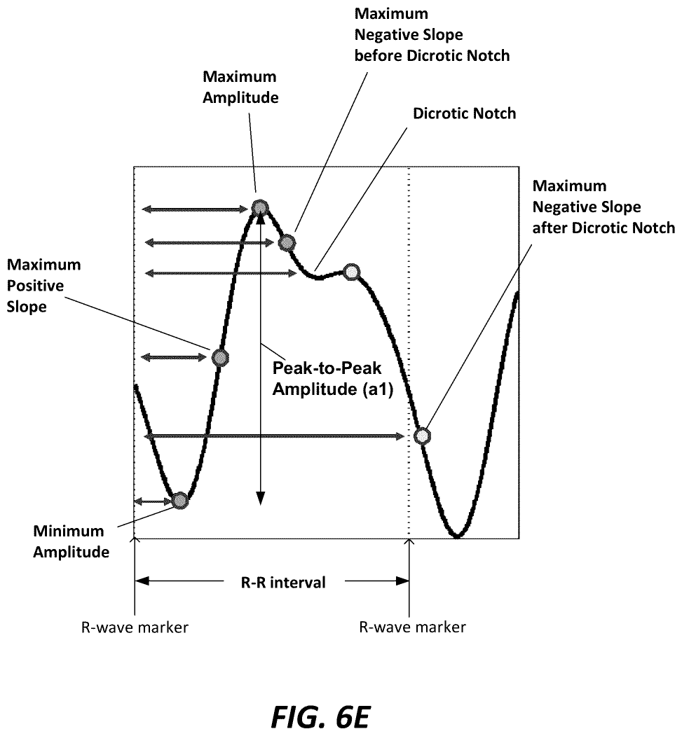

The implantable PPG sensor 303 obtains a PPG signal that after filtering is similar to signal 104 shown in FIGS. 1A-1C, that pulsates over the cardiac cycle. Modulation of the signal occurs because arteries distend as the pressure wave created by the heart's pumping mechanism reaches the sensor site. Such a signal can be filtered and/or amplified as appropriate, e.g., to remove respiratory effects on the signal, and the like. Additionally, the signal can be digitized using an analog to digital converter. Exemplary techniques for performing filtering and other processing of a PPG signal (or other plethysmography signal) are explained with reference to FIGS. 5 and 6A-6E.

For much of above description, it has been assumed that the plethysmography sensor used to produce a plethysmography signal is a PPG sensor. Thus, the plethysmography signal has often been referred to as a PPG signal. However, it should be noted that other types of plethysmography sensors can alternatively be used. Thus, embodiments of the present technology should not be limited to use with PPG sensors and PPG signals. For example, electrodes of the various leads discussed below can be used to obtain an IPG signal, and the IPG signal can be used in place of the PPG signal. Details of exemplary implantable sensors that produce an impedance plethysmography signals are disclosed, e.g., in U.S. Pat. Nos. 4,674,518, 4,686,987 and 5,334,222 (all to Salo), which are incorporated herein by reference.

Still referring to FIG. 3, to sense atrial cardiac signals and to provide right atrial chamber stimulation therapy, the device 310 is coupled to an implantable right atrial lead 320 having at least an atrial tip electrode 322, which typically is implanted in the patient's right atrial appendage. To sense left atrial and ventricular cardiac signals and to provide left-chamber pacing therapy, the device 310 is coupled to a “coronary sinus” lead 324 designed for placement in the “coronary sinus region” via the coronary sinus for positioning a distal electrode adjacent to the left ventricle and/or additional electrode(s) adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus.

Accordingly, an exemplary coronary sinus lead 324 is designed to receive atrial and ventricular cardiac signals and to deliver left ventricular pacing therapy using at least a left ventricular tip electrode 326, left atrial pacing therapy using at least a left atrial ring electrode 327, and shocking therapy using at least a left atrial coil electrode 328.

The device 310 is also shown in electrical communication with the patient's heart 312 by way of an implantable right ventricular lead 330 having, in this embodiment, a right ventricular tip electrode 332, a right ventricular ring electrode 334, a right ventricular (RV) coil electrode 336, and an SVC coil electrode 338. Typically, the right ventricular lead 330 is transvenously inserted into the heart 312 so as to place the right ventricular tip electrode 332 in the right ventricular apex so that the RV coil electrode 336 will be positioned in the right ventricle and the SVC coil electrode 338 will be positioned in the superior vena cava. Accordingly, the right ventricular lead 330 is capable of receiving cardiac signals and delivering stimulation in the form of pacing and shock therapy to the right ventricle.

FIG. 4 will now be used to provide some exemplary details of the components of the implantable devices 310. Referring now to FIG. 4, the implantable devices 310, and alternative versions thereof, can include a microcontroller 460. As is well known in the art, the microcontroller 460 typically includes a microprocessor, or equivalent control circuitry, and can further include RAM and/or ROM memory, logic and timing circuitry, state machine circuitry and/or I/O circuitry. Typically, the microcontroller 460 includes the ability to process or monitor input signals (data) as controlled by a program code stored in a designated block of memory. The details of the design of the microcontroller 460 are not critical to the present technology. Rather, any suitable microcontroller 460 can be used to carry out the functions described herein. The use of microprocessor-based control circuits for performing timing and data analysis functions are well known in the art. In specific embodiments of the present technology, the microcontroller 460 performs some or all of the steps associated with determining estimating changes in arterial blood volume and changes therein, and controlling response thereto.

Representative types of control circuitry that may be used with embodiments of the present technology include the microprocessor-based control system of U.S. Pat. No. 4,940,052 (Mann et. al.) and the state-machines of U.S. Pat. No. 4,712,555 (Sholder) and U.S. Pat. No. 4,944,298 (Sholder). For a more detailed description of the various timing intervals used within the pacing device and their inter-relationship, see U.S. Pat. No. 4,788,980 (Mann et. al.). The '052,'555,'298 and '980 patents are incorporated herein by reference.

Depending on implementation, the device 310 can be capable of treating both fast and slow arrhythmias with stimulation therapy, including pacing, cardioversion and defibrillation stimulation. While a particular multi-chamber device is shown, this is for illustration purposes only, and one of skill in the art could readily duplicate, eliminate or disable the appropriate circuitry in any desired combination to provide a device capable of treating the appropriate chamber(s) with pacing, cardioversion and defibrillation stimulation.

The housing 340, shown schematically in FIG. 4, is often referred to as the “can”, “case” or “case electrode” and may be programmably selected to act as the return electrode for all “unipolar” modes. The housing 340 may further be used as a return electrode alone or in combination with one or more of the coil electrodes, 328, 336 and 338, for shocking purposes. The housing 340 can further include a connector (not shown) having a plurality of terminals, 442, 444, 446, 448, 452, 454, 456, and 458 (shown schematically and, for convenience, the names of the electrodes to which they are connected are shown next to the terminals). As such, to achieve right atrial sensing and pacing, the connector includes at least a right atrial tip terminal (AR TIP) 442 adapted for connection to the atrial tip electrode 322.

To achieve left atrial and ventricular sensing, pacing and shocking, the connector includes at least a left ventricular tip terminal (V_{L} TIP) 444, a left atrial ring terminal (A_{L} RING) 446, and a left atrial shocking terminal (A_{L} COIL) 448, which are adapted for connection to the left ventricular tip electrode 326, the left atrial ring electrode 327, and the left atrial coil electrode 328, respectively.

To support right ventricle sensing, pacing and shocking, the connector further includes a right ventricular tip terminal (V_{R} TIP) 452, a right ventricular ring terminal (V_{R} RING) 454, a right ventricular shocking terminal (R_{V} COIL) 456, and an SVC shocking terminal (SVC COIL) 458, which are adapted for connection to the right ventricular tip electrode 332, right ventricular ring electrode 334, the RV coil electrode 336, and the SVC coil electrode 338, respectively.

An atrial pulse generator 470 and a ventricular pulse generator 472 generate pacing stimulation pulses for delivery by the right atrial lead 320, the right ventricular lead 330, and/or the coronary sinus lead 324 via an electrode configuration switch 474. It is understood that in order to provide stimulation therapy in each of the four chambers of the heart, the atrial and ventricular pulse generators, 470 and 472, may include dedicated, independent pulse generators, multiplexed pulse generators, or shared pulse generators. The pulse generators, 470 and 472, are controlled by the microcontroller 460 via appropriate control signals, 476 and 478, respectively, to trigger or inhibit the stimulation pulses.

The microcontroller 460 further includes timing control circuitry 479 which is used to control pacing parameters (e.g., the timing of stimulation pulses) as well as to keep track of the timing of refractory periods, noise detection windows, evoked response windows, alert intervals, marker channel timing, etc., which is well known in the art. Examples of pacing parameters include, but are not limited to, atrio-ventricular delay, interventricular delay and interatrial delay.

The switch bank 474 includes a plurality of switches for connecting the desired electrodes to the appropriate I/O circuits, thereby providing complete electrode programmability. Accordingly, the switch 474, in response to a control signal 480 from the microcontroller 460, determines the polarity of the stimulation pulses (e.g., unipolar, bipolar, etc.) by selectively closing the appropriate combination of switches (not shown) as is known in the art.

Atrial sensing circuits 482 and ventricular sensing circuits 484 may also be selectively coupled to the right atrial lead 320, coronary sinus lead 324, and the right ventricular lead 330, through the switch 474 for detecting the presence of cardiac activity in each of the four chambers of the heart. Accordingly, the atrial (ATR. SENSE) and ventricular (VTR. SENSE) sensing circuits, 482 and 484, may include dedicated sense amplifiers, multiplexed amplifiers, or shared amplifiers. The switch 474 determines the “sensing polarity” of the cardiac signal by selectively closing the appropriate switches, as is also known in the art. In this way, the clinician may program the sensing polarity independent of the stimulation polarity.

Each sensing circuit, 482 and 484, preferably employs one or more low power, precision amplifiers with programmable gain and/or automatic gain control, band-pass filtering, and a threshold detection circuit, as known in the art, to selectively sense the cardiac signal of interest. The automatic gain control enables the device 310 to deal effectively with the difficult problem of sensing the low amplitude signal characteristics of atrial or ventricular fibrillation. Such sensing circuits, 482 and 484, can be used to determine cardiac performance values used in the present technology. Alternatively, an automatic sensitivity control circuit may be used to effectively deal with signals of varying amplitude.

The outputs of the atrial and ventricular sensing circuits, 482 and 484, are connected to the microcontroller 460 which, in turn, are able to trigger or inhibit the atrial and ventricular pulse generators, 470 and 472, respectively, in a demand fashion in response to the absence or presence of cardiac activity, in the appropriate chambers of the heart. The sensing circuits, 482 and 484, in turn, receive control signals over signal lines, 486 and 488, from the microcontroller 460 for purposes of measuring cardiac performance at appropriate times, and for controlling the gain, threshold, polarization charge removal circuitry (not shown), and timing of any blocking circuitry (not shown) coupled to the inputs of the sensing circuits, 482 and 486.

For arrhythmia detection, the device 310 includes an arrhythmia detector 462 that utilizes the atrial and ventricular sensing circuits, 482 and 484, to sense cardiac signals to determine whether a rhythm is physiologic or pathologic. The timing intervals between sensed events (e.g., P-waves, R-waves, and depolarization signals associated with fibrillation) can be classified by the microcontroller 460 by comparing them to a predefined rate zone limit (i.e., bradycardia, normal, low rate VT, high rate VT, and fibrillation rate zones) and various other characteristics (e.g., sudden onset, stability, physiologic sensors, and morphology, etc.) in order to assist with determining the type of remedial therapy that is needed (e.g., bradycardia pacing, anti-tachycardia pacing, cardioversion shocks or defibrillation shocks, collectively referred to as “tiered therapy”). Additionally, the arrhythmia detector 462 can perform arrhythmia discrimination, e.g., using measures of arterial blood pressure determined in accordance with embodiments of the present technology. The arrhythmia detector 462 can be implemented within the microcontroller 460, as shown in FIG. 4. Thus, this detector 462 can be implemented by software, firmware, or combinations thereof. It is also possible that all, or portions, of the arrhythmia detector 462 can be implemented using hardware. Further, it is also possible that all, or portions, of the ischemia detector 462 can be implemented separate from the microcontroller 460.

In accordance with an embodiment of the present technology, the implantable device 310 includes an arterial blood pressure monitor 469. The arterial blood pressure monitor 469 can be used to monitor the patient's arterial blood pressure and changes therein using the techniques described above. Such techniques can include, e.g., detecting one or more feature(s) of a signal indicative of cardiac activity, detecting one or more feature(s) of a plethysmography signal indicative of changes in arterial blood volume, and determining time(s) between the feature(s) of the signal indicative of cardiac activity and the feature(s) of the plethysmography signal. Such techniques also include estimating arterial blood pressure and changes therein based on such time(s). The arterial blood pressure monitor 469 can also configured to monitor changes in the patient's arterial blood pressure over a day and/or other lengths of time. Additionally, the arterial blood pressure monitor can cause the storing, within the implantable system (e.g., in memory 494), of information indicative of the monitored arterial blood pressure and changes therein so that the stored information is available for transfer to a non-implanted system. Additionally, based on the estimates of the patient's arterial blood pressure and changes therein, the monitor 469 can trigger an alert, therapy and/or adjust therapy, including but not limited to pacing therapy. For another example, trending of the patient's arterial blood pressure can be tracked and used to trigger an alert.

The arterial blood pressure monitor 469 can be implemented within the microcontroller 460, as shown in FIG. 4, and can be implemented by software, firmware, or combinations thereof. It is also possible that all, or portions of arterial blood pressure monitor 469 can be implemented separate from microcontroller 460.

The implantable device 310 can also include a pacing controller 466, which can adjust a pacing rate, pacing intervals and/or pacing configuration based on estimates of arterial blood pressure and/or changes therein, in accordance with embodiments of the present technology. The pacing controller 466 can be implemented within the microcontroller 460, as shown in FIG. 4. Thus, the pacing controller 466 can be implemented by software, firmware, or combinations thereof. It is also possible that all, or portions, of the pacing controller 466 can be implemented using hardware. Further, it is also possible that all, or portions, of the pacing controller 466 can be implemented separate from the microcontroller 460.

The implantable device can also include a medication pump 403, which can deliver medication to a patient if the patient's arterial blood pressure falls above, below, or outside certain thresholds or ranges. Information regarding exemplary implantable medication pumps may be found in U.S. Pat. No. 4,731,051 (Fischell) and in U.S. Pat. No. 4,947,845 (Davis), both of which are incorporated by reference herein.

Still referring to FIG. 4, cardiac signals are also applied to the inputs of an analog-to-digital (A/D) data acquisition system 490. The data acquisition system 490 can be configured to acquire various signal, including but not limited to, EGM, PPG and IPG signals, convert the raw analog data into a digital signal, and store the digital signals for later processing and/or telemetric transmission to an external device 402. The data acquisition system 490 can be coupled to the right atrial lead 320, the coronary sinus lead 324, and the right ventricular lead 330 through the switch 474 to sample cardiac signals across any pair of desired electrodes.

The data acquisition system 490 can be coupled to the microcontroller 460, or other detection circuitry, for detecting an evoked response from the heart 312 in response to an applied stimulus, thereby aiding in the detection of “capture”. Capture occurs when an electrical stimulus applied to the heart is of sufficient energy to depolarize the cardiac tissue, thereby causing the heart muscle to contract. The microcontroller 460 detects a depolarization signal during a window following a stimulation pulse, the presence of which indicates that capture has occurred. The microcontroller 460 enables capture detection by triggering the ventricular pulse generator 472 to generate a stimulation pulse, starting a capture detection window using the timing control circuitry 479 within the microcontroller 460, and enabling the data acquisition system 490 via control signal 492 to sample the cardiac signal that falls in the capture detection window and, based on the amplitude, determines if capture has occurred.

The implementation of capture detection circuitry and algorithms are well known. See for example, U.S. Pat. No. 4,729,376 (Decote, Jr.); U.S. Pat. No. 4,708,142 (Decote, Jr.); U.S. Pat. No. 4,686,988 (Sholder); U.S. Pat. No. 4,969,467 (Callaghan et. al.); and U.S. Pat. No. 5,350,410 (Mann et. al.), which patents are hereby incorporated herein by reference. The type of capture detection system used is not critical to the present technology.

The microcontroller 460 is further coupled to the memory 494 by a suitable data/address bus 496, wherein the programmable operating parameters used by the microcontroller 460 are stored and modified, as required, in order to customize the operation of the implantable device 310 to suit the needs of a particular patient. Such operating parameters define, for example, pacing pulse amplitude, pulse duration, electrode polarity, rate, sensitivity, automatic features, arrhythmia detection criteria, and the amplitude, waveshape and vector of each shocking pulse to be delivered to the patient's heart 312 within each respective tier of therapy. The memory 494 can also store data including information about the patient's arterial blood pressure, cardiovascular risk and/or disease progression.

The operating parameters of the implantable device 310 may be non-invasively programmed into the memory 494 through a telemetry circuit 401 in telemetric communication with an external device 402, such as a programmer, transtelephonic transceiver, or a diagnostic system analyzer. The telemetry circuit 401 can be activated by the microcontroller 460 by a control signal 406. The telemetry circuit 401 advantageously allows EGM and status information relating to the operation of the device 310 (as contained in the microcontroller 460 or memory 494) to be sent to the external device 402 through an established communication link 404. The telemetry circuit can also be used to transmit arterial blood pressure data to the external device 402.

For examples of telemetry devices, see U.S. Pat. No. 4,809,697, entitled “Interactive Programming and Diagnostic System for use with Implantable Pacemaker” (Causey, Ill et al.); U.S. Pat. No. 4,944,299, entitled “High Speed Digital Telemetry System for Implantable Device” (Silvian); and U.S. Pat. No. 6,275,734 entitled “Efficient Generation of Sensing Signals in an Implantable Medical Device such as a Pacemaker or ICD” (McClure et al.), which patents are hereby incorporated herein by reference.

The implantable device 310 additionally includes a battery 411 which provides operating power to all of the circuits shown in FIG. 4. If the implantable device 310 also employs shocking therapy, the battery 411 should be capable of operating at low current drains for long periods of time, and then be capable of providing high-current pulses (for capacitor charging) when the patient requires a shock pulse. The battery 411 should also have a predictable discharge characteristic so that elective replacement time can be detected.

The implantable device 310 is also shown as including an activity and/or posture sensor 415. Such a sensor 415 can be a simple one dimensional sensor that converts mechanical motion into a detectable electrical signal, such as a back electromagnetic field (BEMF) current or voltage, without requiring any external excitation. Alternatively, the sensor 415 can measure multi-dimensional activity information, such as two or more of acceleration, direction, posture and/or tilt. Examples of multi-dimensional activity sensors include, but are not limited to: the three dimensional accelerometer-based position sensor disclosed in U.S. Pat. No. 6,658,292 to Kroll et al., which is incorporated herein by reference; the AC/DC multi-axis accelerometer disclosed in U.S. Pat. No. 6,466,821 to Pianca et al., which in incorporated herein by reference; and the commercially available precision dual-axis accelerometer model ADXL203 and three-axis accelerometer model ADXL346, both available from Analog Devices of Norwood, Massachusetts. Exemplary uses of the activity/posture sensor 415 were discussed above with reference to FIG. 2A.

The implantable device 310 can also include a magnet detection circuitry (not shown), coupled to the microcontroller 460. It is the purpose of the magnet detection circuitry to detect when a magnet is placed over the implantable device 310, which magnet may be used by a clinician to perform various test functions of the implantable device 310 and/or to signal the microcontroller 460 that the external programmer 402 is in place to receive or transmit data to the microcontroller 460 through the telemetry circuits 401.

As further shown in FIG. 4, the device 310 is also shown as having an impedance measuring and processing circuit 413 which is enabled by the microcontroller 460 via a control signal 414 and can be used for obtaining many types of bodily and intracardiac impedances, including a network of single- or multi-vector impedance measurements. Such impedance measurements can be used, e.g., for trending many kinds of physiological variables, and can also be used for detection of air movement in and out of the lungs, blockage of airways, lead impedance surveillance during acute and chronic phases for proper lead positioning or dislodgement; lead integrity by detecting insulation abrasion, operable electrodes, and automatically switching to an operable pair if dislodgement occurs; measuring respiration or minute ventilation; measuring thoracic impedance for determining shock thresholds; detecting when the device has been implanted; measuring cardiac stroke volume; detecting the opening of heart valves; and so forth. The impedance measuring and processing circuit 413 can also be used to produce an IPG signal. The impedance measuring circuit 413 may be coupled to the switch 474 so that any desired electrodes may be used, and networks of vectors can be selected.

In the case where the implantable device 310 is also intended to operate as an implantable cardioverter/defibrillator (ICD) device, it should detect the occurrence of an arrhythmia, and automatically apply an appropriate electrical shock therapy to the heart aimed at terminating the detected arrhythmia. To this end, the microcontroller 460 further controls a shocking circuit 416 by way of a control signal 418. The shocking circuit 416 generates shocking pulses of low (up to 0.5 Joules), moderate (0.5-10 Joules), or high energy (11 to 40 Joules), as controlled by the microcontroller 460. Such shocking pulses are applied to the patient's heart 312 through at least two shocking electrodes, and as shown in this embodiment, selected from the left atrial coil electrode 328, the RV coil electrode 336, and/or the SVC coil electrode 338. As noted above, the housing 340 may act as an active electrode in combination with the RV electrode 336, or as part of a split electrical vector using the SVC coil electrode 338 or the left atrial coil electrode 328 (i.e., using the RV electrode as a common electrode).

The above described implantable device 310 was described as an exemplary pacing device. One or ordinary skill in the art would understand that embodiments of the present technology can be used with alternative types of implantable devices. Accordingly, embodiments of the present technology should not be limited to use only with the above described device. For an example, it is possible that embodiments of the present technology described herein, e.g., with reference to FIG. 2A, be implemented by an IMD that does not provide any type of therapy, but rather, is only capable of performing monitoring. An example of such an IMD is an insertable cardiac monitor (ICM).

### Example Processing of Plethysmography Signals

Photoplethysmography (PPG) and other types of plethysmography signals show changes in a patient's arterial system as a result of the patient's heart contracting, and such signals are indicative of changes in arterial blood volume. A PPG signal can be obtained using a PPG sensor, which as explained above, can be an optical sensor including a light source and a light detector. An IPG signal can be obtained using an IPG sensor, which as explained above, can include electrodes and circuitry used to measure the impedance between such electrodes. One or more such electrodes can be located on one or more leads, and/or a mechanical housing of an implanted device can act as one of the electrodes.

FIGS. 5 and 6A-6E will now be used to describe exemplary embodiments for obtaining a PPG signal and detecting features of the PPG signal. Similar techniques can be used to obtain an IPG signal or other plethysmography signal and detect features of the IPG signal or other plethysmography signal. Referring to FIG. 5, at step 502 a PPG signal is recorded. Recording of a PPG signal may be triggered, e.g., on an R wave, based on respiratory cycle, based on activity levels, etc. An exemplary raw PPG signal recorded over 20 second is shown in FIG. 6A.

At step 504, the PPG signal is filtered to remove respiratory noise, motion artifact, baseline drift, etc. For example, the signal can be band-pass filtered so that the passband is from about 0.7 to 10 Hz, although other pass bands can be used. FIG. 6B shows the raw PPG signal of FIG. 6A, after being band-passed filtered using a passband of about 0.7 to 10 Hz. As can be appreciated from FIG. 6B, most of the respiration signal and high frequency noise is removed by the filtering.

At step 506, an outlier removal process is performed, to remove “bad” heart beats. In an embodiment, the outlier removal can be accomplished by grouping a plurality (e.g., 20) consecutive heart beats, determining a mean of the filtered PPG signal for the plurality of heart beats, and then comparing the determined mean to individual cycles of the filtered PPG signal. Further, outlier removal can be performed by removing each cardiac cycle of the filtered PPG signal that deviates by at least a threshold amount (e.g., 3 or some other number of standard deviations) from the mean of the PPG signal for the plurality of consecutive beats. FIG. 6C show the filtered signal of FIG. 6B with R-wave markers added (shows as dashed vertical lines). FIG. 6D shows the filtered signal of FIGS. 6B and 6C with 3 “bad” beats removed as a result of an outlier removal process.

Still referring to FIG. 5, at step 508, the cycles of the PPG signal remaining after the outlier removal step are then ensemble averaged. The result is an average representation of the PPG signal for the plurality of consecutive beats, with noise and “bad” beats removed. FIG. 6E shows an exemplary ensemble averaged PPG signal.

Thereafter, features of the PPG signal can be detected from the ensemble-averaged PPG signal. For example, as indicated at steps 510 and 512, the first derivative of the ensemble-averaged PPG signal can be determined, and the location of the maximum positive slope of the ensemble-averaged PPG signal can be detected by determining the maximum of the first derivative. Further, since it is believed that the maximum positive slope cannot be more than 70% of an R−R interval away from an R-wave, if the location of the maximum positive slope is not within 70% of an R−R interval away from an R wave, a maximum positive slope detection can be determined to be bad, and not be used.

As indicated at steps 514 and 516, the second derivative of the ensemble averaged PPG signal can be determined to find local minima and maxima. The locations of a maximum and a minimum are where the first derivative is equal to zero. The second derivative can be used to determine if a specific location is a maximum or a minimum. More specifically, if the second derivative is positive, then the point is at a minimum. If the second derivative is negative at a point, then the point is a maximum. The local minimum and local maximum that are closest to the maximum positive slope are the minimum and maximum amplitudes of the signal, which can be used, e.g., to determine the peak-to-peak amplitude of the ensemble averaged PPG signal. Further, as indicated at step 518, the maximum negative slope can be determined by identifying, from the first derivative, the local maximum that occurs after the maximum of the averaged PPG signal, but before the subsequent R-wave. As indicated at step 520, from the second derivative, the dicrotic notch can be identified by identifying the local minimum following the maximum of the averaged PPG signal, but before the subsequent R-wave. FIG. 6E shows examples of various features that can be detected. As shown in FIG. 6E a maximum downward slope can be detected prior to the dicrotic notch, as well as after the dicrotic notch.

Alternative techniques for detecting features of a PPG signal (or other plethysmography signal) can be used, such as, but not limited to, techniques that rely on template matching, wavelets, neural networks, Fast Fourier Transform (FFT) and/or time warping. Alternatively, or additionally, techniques for detecting features of a PPG signal (or other plethysmography signal) can utilize respiratory cycles and R−R intervals.

In certain embodiments, since the presence of the dicrotic notch comes and goes under different conditions, monitoring such conditions can use the presence of the dicrotic notch as a binary feature.

In the above description, embodiments of the present technology were typically described as being directed to IMDs and methods for use with IMDs. It is also possible that one or more non-implanted devices, such as a wrist worn device and/or a chest worn device, or the like, may be used to perform the methods described herein, e.g., with reference to FIG. 2A. For example, such an external device (e.g., a wearable) can store the various calibration factors, obtain a signal (e.g., an electrocardiogram (ECG) signal) indicative of electrical activity of the patient's heart using non-implanted skin electrodes, obtain a signal (e.g., a PPG signal) indicative of changes in arterial blood volume of the patient using a non-implanted PPG sensor (e.g., an optical sensor), and determine a PAT value from such signals (e.g., using a processor of the non-implanted device). Additionally, a non-implanted device can determine a current posture and/or a current activity level of the patient, e.g., using an accelerometer or gyroscope of the wearable device. Additionally, or alternatively, the non-implanted device can determine a current HR of the patient based on a sensed ECG signal. The non-implanted device can also identify one or more of the stored calibration factors that correspond the current posture, the current activity level, and/or the current HR of the patient, or a combination of at least two of the current posture, the current activity level and the current HR of the patient. The non-implanted device can also determine one or more values indicative of the patient's arterial blood pressure based on the PAT value and the one or more identified calibration factors. Examples of wrist worn devices that would be capable of performing such embodiments, and which have electrodes for sensing an ECG signal and a PPG sensor for sensing a PPG signal, include the Apple Watch (TM) available from Apple Inc., headquartered in Cupertino, California, and wearable devices available from Fitbit, headquartered in San Francisco, California, just to name a few.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, it is noted that the term “based on” as used herein, unless stated otherwise, should be interpreted as meaning based at least in part on, meaning there can be one or more additional factors upon which a decision or the like is made. For example, if a decision is based on the results of a comparison, that decision can also be based on one or more other factors in addition to being based on results of the comparison.

Embodiments of the present technology have been described above with the aid of functional building blocks illustrating the performance of specified functions and relationships thereof. The boundaries of these functional building blocks have often been defined herein for the convenience of the description. Alternate boundaries can be defined so long as the specified functions and relationships thereof are appropriately performed. Any such alternate boundaries are thus within the scope and spirit of the claimed embodiments. For example, it would be possible to combine or separate some of the steps shown in FIG. 2A. Further, it is possible to change the order of some of the steps shown in FIG. 2A, without substantially changing the overall events and results. For another example, it is possible to change the boundaries of some of the blocks shown in FIG. 4.

The previous description of the preferred embodiments is provided to enable any person skilled in the art to make or use the embodiments of the present technology. While the present technology has been particularly shown and described with reference to preferred embodiments thereof, it will be understood by those skilled in the art that various changes in form and details may be made therein without departing from the spirit and scope of the present technology.

What is claimed is:

1. A method, comprising:
storing a plurality of calibrations factors, each of which is associated with a respective one of a plurality of different postures, or a respective one of a plurality of different activity levels, or a respective one of a plurality of different heart rate (HR) ranges, or a respective one of a plurality of different combinations of at least two of postures, activity levels and HR ranges for a patient;
obtaining a signal indicative of activity of the patient's heart;
obtaining a signal indicative of changes in arterial blood volume of the patient;
determining a pulse arrival time (PAT) value by determining a time from a feature of the signal indicative of activity of the patient's heart to a feature of the signal indicative of changes in arterial blood volume of the patient;
determining at least one of a current posture, or a current activity level, or a current HR of the patient;
identifying one or more of the stored calibration factors that correspond to at least one of the current posture, or the current activity level, or the current HR of the patient, or a combination of at least two of the current posture, the current activity level and the current HR of the patient; and
determining one or more values indicative of the patient's arterial blood pressure based on the PAT value and the one or more of the stored calibration factors identified at the identifying step.

2. The method of claim 1, further comprising:
at least one of triggering or adjusting therapy based on at least one of the one or more values indicative of the patient's arterial blood pressure.

3. The method of claim 1, wherein:
the obtaining the signal indicative of activity of the patient's heart comprises sensing one of an electrogram (EGM) signal, an electrocardiogram (ECG) signal, or a heart sounds (HS) signal; and
the obtaining the signal indicative of changes in arterial blood volume of the patient comprises sensing one of a photoplethysmography (PPG) signal or an impedance plethysmography (IPG) signal.

4. The method of claim 1, wherein:
the determining at least one of the current posture, or the current activity level, or the current HR of the patient comprises determining the current posture of the patient;
the identifying one or more of the stored calibration factors comprises identifying one or more stored calibration factors that correspond to the current posture of the patient; and
the determining the one or more values indicative of the patient's arterial blood pressure comprises determining the one or more values indicative of the patient's arterial blood pressure based on the PAT value and at least one of the one or more stored calibration factors that correspond to the current posture of the patient.

5. The method of claim 1, wherein:
the determining at least one of the current posture, or the current activity level, or the current HR of the patient comprises determining the current activity level of the patient;
the identifying one or more of the stored calibration factors comprises identifying one or more stored calibration factors that correspond to the current activity level of the patient; and
the determining the one or more values indicative of the patient's arterial blood pressure comprises determining the one or more values indicative of the patient's arterial blood pressure based on the PAT value and at least one of the one or more stored calibration factors that correspond to the current activity level of the patient.

6. The method of claim 1, wherein:
the determining at least one of the current posture, or the current activity level, or the current HR of the patient comprises determining the current HR of the patient;
the identifying one or more of the stored calibration factors comprises identifying one or more stored calibration factors that correspond to the current HR of the patient; and
the determining the one or more values indicative of the patient's arterial blood pressure comprises determining the one or more values indicative of the patient's arterial blood pressure based on the PAT value and at least one of the one or more stored calibration factors that correspond to the current HR of the patient.

7. The method of claim 1, wherein:
the determining at least one of the current posture, the current activity level or the current HR of the patient comprises determining both the current posture and the current HR of the patient;
the identifying one or more of the stored calibration factors comprises identifying one or more stored calibration factors that correspond to a combination of the current posture and the current HR of the patient; and
the determining the one or more values indicative of the patient's arterial blood pressure comprises determining the one or more values indicative of the patient's arterial blood pressure based on the PAT value and at least one of the one or more stored calibration factors that correspond to the combination of the current posture and the current HR of the patient.

8. The method of claim 1, wherein:
the determining at least one of the current posture, the current activity level or the current HR of the patient comprises determining both the current activity level and the current HR of the patient;
the identifying one or more of the stored calibration factors comprises identifying one or more stored calibration factors that correspond to a combination of the current activity level and the current HR of the patient; and
the determining the one or more values indicative of the patient's arterial blood pressure comprises determining the one or more values indicative of the patient's arterial blood pressure based on the PAT value and at least one of the one or more stored calibration factors that correspond to the combination of the current activity level and the current HR of the patient.

9. The method of claim 1, wherein:
the determining at least one of the current posture, the current activity level or the current HR of the patient comprises determining both the current posture and the current activity level of the patient;
the identifying one or more of the stored calibration factors comprises identifying one or more stored calibration factors that correspond to a combination of the current posture and the current activity level of the patient; and
the determining the one or more values indicative of the patient's arterial blood pressure comprises determining the one or more values indicative of the patient's arterial blood pressure based on the PAT value and at least one of the one or more stored calibration factors that correspond to the combination of the current posture and the current activity level of the patient.

10. The method of claim 1, wherein the one or more values indicative of the patient's arterial blood pressure comprises one or more of the following:
a value indicative of the patient's systolic pressure (SP);
a value indicative of the patient's diastolic pressure (DP);
a value indicative of the patient's pulse pressure (PP); or
a value indicative of the patient's mean arterial (MAP).

11. The method of claim 1, further comprising:
obtaining one or more values indicative of the patient's arterial blood pressure from a non-implanted device, and based thereon, determining or updating one or more of the stored calibrations factors; and
wherein the one or more of the stored calibrations factors, which is/are updated based on the one or more values indicative of the patient's arterial blood pressure obtained from the non-implanted device, is dependent on at least one of the current posture, the current activity level, or the current HR of the patient, when the one or more values indicative of the patient's arterial blood pressure was obtained from the non-implanted device.

12. The method of claim 1, wherein:
the obtaining the signal indicative of changes in arterial blood volume of the patient comprises sensing both a photoplethysmography (PPG) signal and an impedance plethysmography (IPG) signal; and
further comprising selecting, based on at least one of the current posture, the current activity level, or the current HR of the patient, which one of the PPG and the IPG signals is to be used for the determining the PAT value, which is used for the determining the one or more values indicative of the patient's arterial blood pressure.

13. The method of claim 1, wherein:
the obtaining the signal indicative of changes in arterial blood volume of the patient comprises sensing both a photoplethysmography (PPG) signal and an impedance plethysmography (IPG) signal;
the determining the PAT value comprises determining a first PAT value using the PPG signal and determining a second PAT value using the IPG signal; and
the determining the one or more values indicative of the patient's arterial blood pressure comprises determining a first value indicative of the patient's arterial blood pressure based on the first PAT value, and determining a second value indicative of the patient's arterial blood pressure based on the second PAT value.

14. The method of claim 13, further comprising:
selecting, based on at least one of the current posture, the current activity level or the current HR of the patient, which one of the first and the second values indicative of the patient's arterial blood pressure is to be at least one of stored or used by the IMD.

15. The method of claim 13, further comprising:
selecting, based on at least one of the current posture, the current activity level or the current HR of the patient, one of a plurality of different ways to determine a weighted average of the first and the second values indicative of the patient's arterial blood pressure; and
at least one of storing or using the weighted average of the first and the second values indicative of the patient's arterial blood pressure.

16. A system, comprising:
memory that stores a plurality of calibrations factors each of which is associated with a respective one of a plurality of different postures, or a respective one of a plurality of different activity levels, or a respective one of a plurality of different heart rate (HR) ranges, or a respective one of a plurality of different combinations of at least two of postures, activity levels and HR ranges for a patient;
electrodes and/or one or more sensors configured to sense a signal indicative of activity of the patient's heart, and configured to sense a signal indicative of changes in arterial blood volume of the patient;
one or more further sensors configured to output one or more signals indicative of at least one of a current posture or a current activity level of the patient; and
one or more controllers configured to determine a pulse arrival time (PAT) value by determining a time from a feature of the signal indicative of activity of the patient's heart to a feature of the signal indicative of changes in arterial blood volume of the patient;
identify one or more of the stored calibration factors that correspond to at least one of the current posture, or the current activity level, or a current HR of the patient, or a combination of at least two of the current posture, the current activity level and the current HR of the patient; and
determine one or more values indicative of the patient's arterial blood pressure based on the PAT value and the identified at least one of the one or more stored calibration factors that correspond to at least one of the current posture, or the current activity level, or the current HR of the patient, or a combination of at least two of the current posture, the current activity level and the current HR of the patient.

17. The system of claim 16, wherein the one or more controllers is/are configured to:
at least one of trigger or adjust therapy based on at least one of the one or more values indicative of the patient's arterial blood pressure.

18. The system of claim 16, wherein:
the signal indicative of activity of the patient's heart comprises one of an electrogram (EGM) signal, an electrocardiogram (ECG) signal or a heart sounds (HS) signal; and
the signal indicative of changes in arterial blood volume of the patient comprises one of a photoplethysmography (PPG) signal or an impedance plethysmography (IPG) signal.

19. The system of claim 16, wherein the one or more values indicative of the patient's arterial blood pressure comprises one or more of the following:
a value indicative of the patient's systolic pressure (SP);
a value indicative of the patient's diastolic pressure (DP);
a value indicative of the patient's pulse pressure (PP); or
a value indicative of the patient's mean arterial (MAP).

20. The system of claim 16, wherein:
the electrodes and/or one or more sensors are used to sense both a photoplethysmography (PPG) signal and an impedance plethysmography (IPG) signal; and
the one or more controllers is/are configured to select, based on at least one of the current posture, the current activity level, or the current HR of the patient, which one of the PPG and the IPG signals is to be used to determine the PAT value, which is used to determine the one or more values indicative of the patient's arterial blood pressure.

21. The system of claim 16, wherein:
the electrodes and/or one or more sensors are used to sense both a photoplethysmography (PPG) signal and an impedance plethysmography (IPG) signal; and
the one or more controllers is/are configured to determine a first PAT value using the PPG signal and determine a second PAT value using the IPG signal; and
determine a first value indicative of the patient's arterial blood pressure based on the first PAT value, and determine a second value indicative of the patient's arterial blood pressure based on the second PAT value.

22. The system of claim 21, wherein the one or more controllers is/are configured to:
select, based on at least one of the current posture, the current activity level, or the current HR of the patient, which one of the first and the second values indicative of the patient's arterial blood pressure is to be at least one of stored or used by the IMD.

23. The system of claim 21, wherein the one or more controllers is/are configured to:
select, based on at least one of the current posture, the current activity level or the current HR of the patient, one of a plurality of different ways to determine a weighted average of the first and the second values indicative of the patient's arterial blood pressure; and
at least one of store or use the weighted average of the first and the second values indicative of the patient's arterial blood pressure.

24. One or more processor readable storage devices having instructions encoded thereon which when executed cause one or more processors to perform a method, the method comprising:
storing a plurality of calibrations factors, each of which is associated with a respective one of a plurality of different postures, or a respective one of a plurality of different activity levels, or a respective one of a plurality of different heart rate (HR) ranges, or a respective one of a plurality of different combinations of at least two of postures, activity levels and HR ranges;
obtaining a signal indicative of activity of the patient's heart;
obtaining a signal indicative of changes in arterial blood volume of the patient;
determining a pulse arrival time (PAT) value by determining a time from a feature of the signal indicative of activity of the patient's heart to a feature of the signal indicative of changes in arterial blood volume of the patient;
determining at least one of a current posture, or a current activity level, or a current HR of the patient;
identifying one or more of the stored calibration factors that correspond to at least one of the current posture, or the current activity level, or the current HR of the patient, or a combination of at least two of the current posture, the current activity level and the current HR of the patient; and
determining one or more values indicative of the patient's arterial blood pressure based on the PAT value and the one or more of the stored calibration factors identified at the identifying step.

25. The one or more processor readable storage devices having instructions encoded thereon of claim 24, wherein the method further comprises:
at least one of triggering or adjusting therapy based on at least one of the one or more values indicative of the patient's arterial blood pressure.
